(19) United States
(12) Patent Application Publication
(10) Pub. No.: US 2020/0058410 A1
(43) Pub. Date: Feb. 20, 2020

(54) METHOD AND APPARATUS FOR IMPROVING SUBJECT TREATMENT AND NAVIGATION RELATED TO A MEDICAL TRANSPORT TELEPRESENCE SYSTEM

(71) Applicant: Medris, LLC, Charlotte, NC (US)

(72) Inventors: George Abraham KHOURI, III, Charlotte, NC (US); Andrew Tyler BLANTON, Charlotte, NC (US)

(21) Appl. No.: 16/102,808

(22) Filed: Aug. 14, 2018

(51) Int. Cl. G16H 80/00; G16H 50/20

(52) U.S. Cl. CPC G16H 80/00; G16H 50/20

(57) ABSTRACT

Methods and apparatus for diagnosing and routing a patient are provided. A telepresence system includes a medical transport; a communications controller configured to connect the medical transport with a remote telehealth specialist; an input device in the medical transport configured to receive at least one input, including a code identification, from medical transport personnel; a microphone configured to generate a first audio signal and transmit the first audio signal to the communications controller; a camera mounted in the medical transport, wherein the camera is configured to generate a first video signal and transmit the first video signal to the communications controller; a navigation analyzer; and at least one display disposed in the medical transport.

### BACKGROUND

Historically, ambulances, helicopters, and other medical transport have had limited treatment options available, often being limited to triage and stabilization of the patient before arriving at a hospital. A problem with these systems is that a patient may require a treatment (e.g., drug or surgery) that is not available in certain emergency facilities. Treatment may, thus, be unacceptably delayed by bringing a patient to the nearest emergency facility without first identifying their underlying condition. Further, many patients do not receive optimal care because they are transported out of their typical network of healthcare providers.

Various methods, apparatus, and systems are configured in a medical transport telepresence system. Applicant has identified many deficiencies and problems associated with existing methods, apparatus, and systems. Through applied effort, ingenuity, and innovation, these identified deficiencies and problems have been solved by developing solutions that are in accordance with the embodiments of the present invention, many examples of which are described in detail herein.

### BRIEF SUMMARY

In general, embodiments of the present invention provide methods, apparatus, systems, computing devices, and/or the like for improving the diagnosis and treatment of a subject, and navigating the subject to the most compatible hospital or clinic.

In accordance with one aspect, a telepresence system for diagnosing and routing a patient is provided. The telepresence system comprises a medical transport at a first location; a communications controller configured to connect the medical transport with a remote telehealth specialist; an input device in the medical transport configured to receive at least one input, including a code identification, from medical transport personnel; a microphone configured to generate a first audio signal and transmit the first audio signal to the communications controller; a camera mounted in the medical transport, wherein the camera is configured to generate a first video signal and transmit the first video signal to the communications controller; a navigation analyzer; and at least one display disposed in the medical transport; wherein the communications controller is configured to: transmit the at least one input via at least one of a first wireless network and a second wireless network; connect the medical transport to the remote telehealth specialist at a second location via each of the first wireless network and the second wireless network, transmit the first video signal from the camera to the remote telehealth specialist via the first wireless network, transmit the first audio signal from the microphone to the remote telehealth specialist via the second wireless network, receive a second video signal from the remote telehealth specialist over the first wireless network; transmit the second video signal to the at least one display in the medical transport; receive a second audio signal from the remote telehealth specialist over the second wireless network; transmit the second audio signal to at least one audio output device in the medical transport; receive a diagnostic indication from the remote telehealth specialist via at least one of the first wireless network and the second wireless network; and transmit the diagnostic indication to the navigation analyzer; wherein the navigation analyzer is configured to: receive at least one of the first location of the medical transport and a current location of the medical transport; receive the diagnostic indication; generate navigation data comprising navigation instructions from the at least one of the first location of the medical transport and the current location of the medical transport to an emergency department based on the diagnostic indication; and transmit the navigation data to the at least one display in the medical transport.

In accordance with another aspect, the code identification indicating the diagnosis of a patient is a code identification for a heart attack.

In accordance with another aspect, the code identification indicating the diagnosis of a patient is a code identification for a cerebrovascular accident.

In accordance with another aspect, the code identification for the cerebrovascular accident is related to a large vessel occlusion.

In accordance with another aspect, the code identification for the cerebrovascular accident is not related to a large vessel occlusion.

In accordance with another aspect, the navigation data to the emergency department is to an outpatient only facility.

In accordance with another aspect, the navigation data to the emergency department is to an inpatient capable facility.

In accordance with another aspect, the navigation data to the emergency department is to a non-invasive treatment facility.

In accordance with another aspect, the navigation data to the emergency department is to an invasive treatment facility.

In accordance with another aspect, first wireless network and the second wireless network each comprises at least one of cellular signals, radio signals, or Wi-Fi signals.

In accordance with another aspect, the emergency department comprises a home hospital network emergency department that is within the patient's home hospital network.

In accordance with another aspect, the emergency department comprises a payor network emergency department that is within the patient's payor network.

In accordance with another aspect, a telepresence system for diagnosing and routing a patient comprising: a medical transport at a first location; a communications controller configured to connect the medical transport with a remote telehealth specialist; an input device in the medical transport configured to receive at least one input, including a code identification, from medical transport personnel; a microphone configured to generate a first audio signal and transmit the first audio signal to the communications controller; a camera mounted in the medical transport, wherein the camera is configured to generate a first video signal and transmit the first video signal to the communications controller; a navigation analyzer; and at least one display disposed in the medical transport; wherein the communications controller is configured to: transmit the at least one input via at least one of a first wireless network and a second wireless network; connect the medical transport to the remote telehealth specialist at a second location via each of the first wireless network and the second wireless network, transmit the first video signal from the camera to the remote telehealth specialist via the first wireless network, transmit the first audio signal from the microphone to the remote telehealth specialist via the second wireless network, receive a second video signal from the remote telehealth specialist over the first wireless network; transmit the second video signal to the at least one display in the medical transport; receive a second audio signal from the remote telehealth specialist over the second wireless network; transmit the second audio signal to at least one audio output device in the medical transport; receive a diagnostic indication from the remote telehealth specialist via at least one of the first wireless network and the second wireless network; and transmit the diagnostic indication to the navigation analyzer wherein the navigation analyzer is configured to: receive at least one of the first location of the medical transport and a current location of the medical transport; receive the code identification; receive the navigation diagnostic indication; generate navigation data comprising navigation instructions from the at least one of the first location of the medical transport and the current location of the medical transport to an emergency department based on the code identification and the diagnostic indication; and transmit the navigation data to the at least one display in the medical transport.

In accordance with another aspect, a method for communicating between a telepresence system at a first location and a remote telehealth specialist at a second location is provided. The method comprises generating a first video signal from a camera mounted in a medical transport at the first location; generating a first audio signal from a microphone mounted in the medical transport; receiving a code identification from an input device mounted in the medical transport; transmitting the first video signal to the remote telehealth specialist at a separate remote location using a first wireless network; transmitting the first audio signal to the remote telehealth specialist at a separate remote location using a second wireless network; transmitting the code identification to the remote telehealth specialist via at least one of the first wireless network and the second wireless network; receiving a second video signal over the first wireless network of the remote telehealth specialist from a camera at the remote location; receiving a second audio signal over the second wireless network of from the remote telehealth specialist from a microphone at the remote location; receiving a diagnostic indication from over either the first or second network from the remote telehealth specialist; displaying the second video signal on a display in the medical transport; playing the second audio signal over a speaker in the medical transport; generating navigation data to an emergency department based on at least one of the code identification or the diagnostic indication; displaying the navigation data on the display in the medical transport.

In accordance with another aspect, a computer system having at least one processor and at least one non-transitory memory including program code is provided. The at least one non-transitory memory and the program code are configured to, with the at least one processor, cause the computer system to at least generate a diagnostic request associated with a subject; transmit the diagnostic request to a remote party; receive a diagnostic indication from the remote party in response to the diagnostic request; determine an emergency department based at least in part on the diagnostic indication; and generate navigation data to the emergency department.

In accordance with another aspect, the subject is in need of treatment for a cerebrovascular accident, and the diagnostic request is associated with the cerebrovascular accident, the at least one non-transitory memory and the program code configured to, with the at least one processor, cause the computer system to further generate an indication to perform at least one of: administering a therapeutically effective amount of tissue plasminogen activator to the subject, or performing a thrombectomy on the subject.

In accordance with another aspect, the diagnostic indication comprises data indicating that the subject has a cerebrovascular accident, and the cerebrovascular accident is not related to large vessel occlusion, wherein generating navigation data based at least in part on the diagnostic indication further comprises: determining a time interval for administering a therapeutically effective amount of tissue plasminogen activator; calculating a time period for transporting the subject to a nearest outpatient only facility and administering the therapeutically effective amount of tissue plasminogen activator in the nearest outpatient only facility; and determining whether the time period is within the time interval.

In accordance with another aspect, the at least one non-transitory memory and the program code are configured to, with the at least one processor, cause the computer system to further determine that the time period is within the time interval, wherein the emergency department is the nearest outpatient only facility.

In accordance with another aspect, the at least one non-transitory memory and the program code are configured to, with the at least one processor, cause the computer system to further determine that the time period is not within the time interval, wherein the emergency department is a nearest inpatient capable facility.

In accordance with another aspect, the diagnostic indication comprises data indicating that the subject has a cerebrovascular accident, and the cerebrovascular accident is related to large vessel occlusion, wherein generating navigation data based at least in part on the diagnostic indication further comprises: determining a time interval for administering a therapeutically effective amount of tissue plasminogen activator; calculating a time period for transporting the subject to a nearest surgical facility and administering the therapeutically effective amount of tissue plasminogen activator in the nearest surgical facility; and determining whether the time period is within the time interval.

In accordance with another aspect, the at least one non-transitory memory and the program code are configured to, with the at least one processor, cause the computer system to further determine that the time period is within the time interval, wherein the emergency department is the nearest surgical facility.

In accordance with another aspect, the at least one non-transitory memory and the program code are configured to, with the at least one processor, cause the computer system to further determine that the time period is not within the time interval, wherein the emergency department is a nearest inpatient capable facility.

In accordance with another aspect, the diagnostic request comprises identification data and medical history data associated with the subject.

In accordance with another aspect, the diagnostic request comprises data indicating a last known well time of the subject.

In accordance with another aspect, transmitting the diagnostic request to a remote party further comprises: receiving a communication request; establishing a first communication link via a first network and a second communication link via a second network, wherein the first network is separated from the second network; transmitting, via the first communication link, a video audio signal to the remote party; and transmitting, via the second communication link, a video image signal to the remote party.

In accordance with another aspect, the at least one non-transitory memory and the program code are configured to, with the at least one processor, cause the computer system to further: receive, via the first communication link, a received video audio signal from the remote party; receive, via the second communication link, a received video image signal from the remote party; and output the received video audio signal and the received video image signal.

In accordance with another aspect, a method of treating a subject in need thereof is provided. The method comprises generating, by one or more configured computing systems, a diagnostic request associated with the subject; transmitting, by the one or more configured computing systems, the diagnostic request to a remote party; receiving, by the one or more configured computing systems, a diagnostic indication from the remote party in response to the diagnostic request; generating, by the one or more configured computing systems, navigation data based at least in part on the diagnostic indication; and transporting the subject to an emergency department based on the navigation data.

In accordance with another aspect, the subject is in need of treatment for a cerebrovascular accident, and the diagnostic request is associated with the cerebrovascular accident. The method further comprises: performing at least one of: administering a therapeutically effective amount of tissue plasminogen activator to the subject, or performing a thrombectomy on the subject.

In accordance with another aspect, the diagnostic indication comprises data indicating that the subject has a cerebrovascular accident, and the cerebrovascular accident is not related to large vessel occlusion, wherein generating navigation data based at least in part on the diagnostic indication further comprises: determining, by the one or more configured computing systems, a time interval for administering a therapeutically effective amount of tissue plasminogen activator; calculating, by the one or more configured computing systems, a time period for transporting the subject to a nearest outpatient only facility and administering the therapeutically effective amount of tissue plasminogen activator in the nearest outpatient only facility; and determining, by the one or more configured computing systems, whether the time period is within the time interval.

In accordance with another aspect, the method further comprises determining that the time period is within the time interval, wherein the emergency department is the nearest outpatient only facility.

In accordance with another aspect, the method further comprises determining that the time period is not within the time interval, wherein the emergency department is a nearest inpatient capable facility.

In accordance with another aspect, the diagnostic indication comprises data indicating that the subject has a cerebrovascular accident, and the cerebrovascular accident is related to large vessel occlusion, wherein generating navigation data based at least in part on the diagnostic indication further comprises: determining, by the one or more configured computing systems, a time interval for administering a therapeutically effective amount of tissue plasminogen activator; calculating, by the one or more configured computing systems, a time period for transporting the subject to a nearest surgical facility and administering the therapeutically effective amount of tissue plasminogen activator in the nearest surgical facility; and determining, by the one or more configured computing systems, whether the time period is within the time interval.

In accordance with another aspect, the method further comprises determining that the time period is within the time interval, wherein the emergency department is the nearest surgical facility.

In accordance with another aspect, the method further comprise determining that the time period is not within the time interval, wherein the emergency department is a nearest inpatient capable facility.

In accordance with another aspect, transmitting the diagnostic request to a remote party further comprises receiving, by the one or more configured computing systems, a communication request; establishing, by the one or more configured computing systems, a first communication link via a first network and a second communication link via a second network, wherein the first network is separated from the second network; transmitting, by the one or more configured computing systems and via the first communication link, a video audio signal to the remote party; and transmitting, by the one or more configured computing systems and via the second communication link, a video image signal to the remote party.

In accordance with another aspect, a computer program product is provided. The computer program product comprising at least one non-transitory computer-readable storage medium having computer-readable program code portions stored therein, the computer-readable program code portions comprising an executable portion configured to: generate a diagnostic request associated with a subject; transmit the diagnostic request to a remote party; receive a diagnostic indication from the remote party in response to the diagnostic request; determine an emergency department based at least in part on the diagnostic indication; and generate navigation data to the emergency department.

In accordance with another aspect, a communication device is provided. The communication device is configured to generate a diagnostic request associated with a subject; transmit the diagnostic request to a remote party; receive a diagnostic indication from the remote party in response to the diagnostic request; determine an emergency department based at least in part on the diagnostic indication; and generate navigation data to the emergency department.

### BRIEF DESCRIPTION OF THE DRAWINGS

Having thus described some embodiments in general terms, references will now be made to the accompanying drawings, which are not drawn to scale, and wherein:

FIG. 1 is an example system architecture schema in accordance with various embodiments of the present invention;

FIG. 2 is an example schematic diagram of an apparatus in accordance with various embodiments of the present invention;

FIGS. 3A, 3B, 3C, 3D, 3E, and 3F are example flow charts illustrating example methods in accordance with various embodiments of the present invention;

FIG. 4 is an example flow chart illustrating example methods in accordance with various embodiments of the present invention;

FIG. 5 is an example flow chart illustrating example methods in accordance with various embodiments of the present invention;

FIGS. 6A, 6B, and 6C are schematic perspective views of a device in accordance with various embodiments of the present invention;

FIG. 7 is a schematic diagram illustrating various concepts in accordance with some embodiments of the present invention;

FIGS. 8-9 are example schematic diagrams of various components in accordance with embodiments of the present invention; and

FIGS. 10A-10C are schematic perspective views of a device in accordance with various embodiments of the present invention.

### DETAILED DESCRIPTION OF VARIOUS EMBODIMENTS

Various embodiments of the present invention now will be described more fully hereinafter with reference to the accompanying drawings, in which some, but not all embodiments of the invention are shown. Indeed, the invention may be embodied in many different forms and should not be construed as limited to the embodiments set forth herein. Rather, these embodiments are provided so that this disclosure will satisfy applicable legal requirements. The term “or” is used herein in both the alternative and conjunctive sense, unless otherwise indicated. The terms “illustrative,” “example,” and “exemplary” are used to be examples with no indication of quality level. Like numbers refer to like elements throughout.

### Overview

Various embodiments of the present invention relate generally to improving medical transport telepresence systems. More specifically, various embodiments of the present invention are related to improving the diagnosis of a subject in need of treatment, and providing improvements on navigating the subject to the most compatible or in-network hospital or clinic. In some embodiments, the network may be home healthcare system for patient, network within patient's insurance coverage, or network designated by patient in advance.

Existing systems that do not incorporate the inventive embodiments discussed herein (“deficient systems”) may be plagued by limitations and inefficiencies, including failing to deliver the subject (e.g., a patient suffering from one or more ailments) to the most compatible hospital or clinic based on the subject's conditions and within a certain time window. However, emergency medical technicians (“EMTs”) are unable to fully or accurately diagnose and treat many ailments, and instead attempt to stabilize the patient and transport them to the nearest emergency room for diagnosis and treatment on-site. This causes increased delays and inefficiencies when the on-site diagnosis determines that the patient needs a drug or procedure that is not administered at that particular emergency room. Likewise, EMTs cannot take every patient to the most sophisticated nearby hospital because the needed assistance may be available at a closer, less comprehensive emergency room and because the sophisticated hospital would quickly be inundated with patients, which would further delay and impair the treatment of all patients. Further, in the instances where there are many compatible sites in a small region of space, deficient systems fail to identify that the most suitable one to care for a patient should be the one that is familiar with that patient or in the network (ACO, hospital system, insurance, etc.).

One example of this navigational problem costing valuable time will be described in the context of cerebrovascular accidents (e.g., strokes). Strokes may be caused by a blockage of the arteries leading to the brain, which is referred to as an ischemic stroke. For example, when a subject is exhibiting signs of a stroke, a diagnosis needs to be made on whether the symptoms are of an ischemic stroke due to large vessel occlusion (“LVO”). Currently, tissue plasminogen activator (“tPA”) is the only FDA-approved treatment agent for ischemic strokes. Prior to administering tPA, a computed tomographic (CT) scan needs to be conducted to make sure that there is no blood in the brain. However, tPA is typically administered to the subject within a certain time window that is established by the health care industry (e.g., 4.5 hours from last known well time). Deficient systems fail to provide accurate diagnosis of the subject in time to administer tPA, thus causing subjects missing this key brain-saving treatment.

Further, subjects who have large vessel occlusion (“LVO”) may require surgical removal of the blood clot (thrombectomy) instead of, or in addition to, tPA. However, not all hospitals have the necessary equipment and personnel to conduct these operations. Moreover, medical transport personnel (e.g., EMTs) are not capable of diagnosing LVO, which requires a skilled specialist, such as a neurologist, to diagnose in the field and requires sophisticated imaging technology (e.g., a computed tomography (CT) scan) without an exam from a trained specialist to confirm. Deficient systems fail to recognize this need or have the capability of differentiating the two situations (e.g., LVO versus non-LVO), and, as a result, EMTs may transport the subjects to the incompatible hospital or clinic. For example, some deficient systems may first deliver subjects to a nearby urgent care clinic and, only after recognizing the need for thrombectomy at the clinic, deliver these subjects to a suitably equipped hospital, sometimes referred to as a hub hospital. This deficient system is wasting unnecessary transportation time and possibly causing irreparable harm to the patient. Moreover, both thrombectomies and tPA have maximum time limits before the treatments are no longer viable. Thus, unnecessary delay may preclude a patient from receiving life-saving treatment.

In contrast, systems structured in accordance with various embodiments of the present invention overcome challenges faced by deficient systems. For example, in some embodiments of the present invention, the systems transmit data related to the subject to a telehealth specialist, and receive diagnostic indications from the telehealth specialist while the subject is still in the medical transport, such as an ambulance. Based on the received diagnostic indications, the systems then programmatically navigate the medical transport to the nearest hospital or clinic that will provide the most compatible treatment to the subject. By doing so, the systems deliver subjects in the most efficient manner so that subjects can receive the most effective treatments. This increase in efficiency corresponds to cost savings and greater availability of medical personnel, which allows the treatment of additional patients.

Various embodiments of the present invention provides an “ambulance navigation system” using telehealth specialist and algorithmic real-time software, which includes ability to identify patient information in the system database and route/navigate certain patients to home hospital and keep patients in their payor network (ACO, Insurance, etc.).

As such, systems structured in accordance with various embodiments of the invention provide specific, technical solutions to technical problems faced by deficient systems, the details of which are described hereinafter.

### Definitions

As used herein, the terms “data,” “content,” “information,” and similar terms may be used interchangeably to refer to data capable of being transmitted, received, and/or stored in accordance with embodiments of the present invention. Thus, use of any such terms should not be taken to limit the spirit and scope of embodiments of the present invention. Further, where a computing device is described herein to receive data from another computing device, it will be appreciated that the data may be received directly from another computing device or may be received indirectly via one or more intermediary computing devices, such as, for example, one or more servers, relays, routers, network access points, base stations, hosts, and/or the like, sometimes referred to herein as a “network,” unless provided otherwise. Similarly, where a computing device is described herein to send data to another computing device, it will be appreciated that the data may be sent directly to another computing device or may be sent indirectly via one or more intermediary computing devices, such as, for example, one or more servers, relays, routers, network access points, base stations, hosts, and/or the like.

The term “user” should be understood to refer to an individual, group of individuals, business, organization, and the like.

The term “database” refers to a collection of data in a computer network that is organized so that its content can easily be accessed, managed, and updated. Databases may contain aggregations of datasets, such as user names, user identification information, and user profiles. Databases may be classified according to its dataset type, such as bibliographic, full text, numeric, and images. Databases may also be classified based on its organizational structure, such as relational database, distributed database, cloud database, Not Only SQL (NoSQL) database, object-oriented database, and graph database.

The term “subject” refers to a person who is subjected to treatment and/or observation. For example, a patient. Information related to the subject may be stored in a medical database. For example, such information may include identification data of the subject (e.g. date of birth) and medical history data (e.g. current and past medical issues of the subject, the subject's family history, and other relevant health information). In this regard, the setup and operations of the medical database must be in compliance with various regulatory mandates, including, for example, the Health Insurance Portability and Accountability Act (“HIPAA”) in the United States.

The term “remote party” refers to a person who is at a distance from a subject. For example, a “specialist” or “telehealth specialist” (or “tele-specialist”/“telespecialist”) is a remote party who is a doctor or specialist and provides diagnosis and/or treatment information of a subject from a distance. In this regard, medical transport, such as an ambulance, may implement a telepresence system, allowing a telehealth specialist to conduct diagnostic and other related medical procedures from a distance utilizing systems structure in accordance with various embodiments of the present invention. For example, a remote neurologist may be a telehealth specialist in the context of a possible stroke. In some embodiments, the remote party may be located at an emergency department, whether or not the subject is ultimately taken to the same emergency department. Further, in some embodiments, the remote party is capable of routing communication requests from a telepresence system such that the request is reached to the most suitable destination. Communications to and from the remote party are encrypted to comply with various requirements, including HIPAA.

The term “medical transport” refers to carriers and vehicles (for example, ambulances, ships, helicopters, planes, etc.) that transport subjects. A “medical transport telepresence system” refers to an integrated communication system that enables communications between the medical transport and remote locations outside the medical transport, including one or more remote parties. The integrated communication system may include one or more channels.

The term “electronic indication” refers to an electronic communication, a video signal, an audio signal, communication link, a display, an electronic mail, a text message, an application alert, a mobile application notification, and/or other type of electronic interface or distribution channel that delivers relation information. For example, in the context of medical transport telepresence system, an electronic indication may indicate information such as emergency type (e.g. trauma, stroke, heart attack) and conditions of the subject (e.g. vital signs such as body temperature, pulse rate, respiration rate, blood pressure, etc.) as well as provide a communication link. The medical transport telepresence system may transmit a diagnostic request to a remote party, such as a telehealth specialist, to conduct various diagnosis based on the electronic indications. In this regard, the telehealth specialist may receive the diagnostic request on one or more computing apparatus, such as smart wearable, smart phone, personal computers desktop computer, etc. The telehealth specialist may then return a diagnostic indication, indicating what disease or condition explains the subject's symptoms and signs, which indication may trigger further action from the systems detailed herein.

“Cerebrovascular accident” (or “stroke”) occurs when blood flow to a part of a subject's brain is suddenly stopped and oxygen cannot get to the affected part. In some cases, the lack of oxygen may be caused by a blocked artery. For example, it may be caused by large vessel occlusion (or “LVO”), including vertebral, basilar, carotid terminus, middle and anterior cerebral arteries. In some cases, it may be caused by the leaking or bursting of a blood vessel. The lack of oxygen may damage or kill brain cells, causing death of the tissues in a part of the brain.

The “last known well time” refers to the time at which the subject was last known to be without the signs and symptoms of a current ailment (e.g., cerebrovascular accident, trauma, myocardial infarction, etc.) or at his or her baseline state of health. In some instances, this last known well time may be approximated, for example, if symptoms begin while the patient is asleep.

“Tissue plasminogen activator” (or “tPA”) is an enzyme that helps dissolve clots in blood vessels. Administering a therapeutically effective amount (an amount sufficient to have therapeutic benefit) of tissue plasminogen activator in a subject having a cerebrovascular accident can provide relief to the symptoms. In this regard, the time frame for administering tPA may be subject to regulatory and/or industry mandates or other requirements. In some instances, tPA must be administered within 3 hours since the last known well time, or up to 4.5 hours in certain eligible patients. Outside the time frame, the brain tissue of the patient having a cerebrovascular accident is likely already dead. As such, administering tPA to a patient outside the tPA time frame may cause a hemorrhage. Similarly, the time frame for conducting thrombectomy may also be subject to regulatory and/or industrial mandates or other requirements, and the time frame for conducting thrombectomy is typically longer than the time frame for administering tPA.

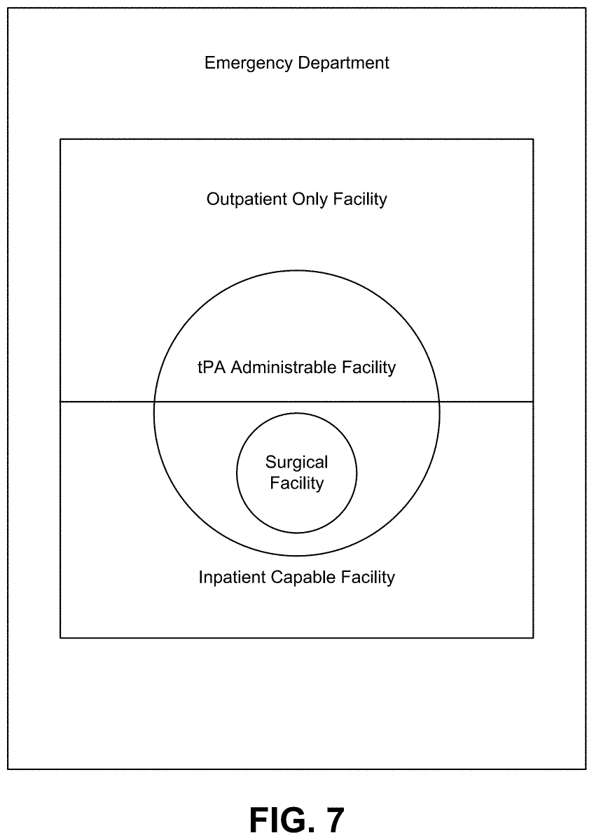

The term “emergency department” refers to a location that provides acute care of patients who are present without prior appointment. As shown in FIG. 7, an emergency department may be an “outpatient only facility” (or “freestanding emergency room (FRSTND)”), where a patient cannot be admitted as inpatient and no overnight stay is provided. An emergency department may be an “inpatient capable facility,” where patient can be admitted as inpatient and stay overnight. An emergency department may be a non-invasive treatment facility, which includes, for example, tPA administrable facilities where tPA may be administered to the subject by a registered nurse of the emergency department after confirmed diagnosis from a physician. An emergency department may be “invasive treatment facility” (or “Thrombectomy Capable Hospital (TCH)”) which includes, for example, surgical or neurosurgical facilities where a physician may conduct thrombectomy. Information related to the surgical and treatment capabilities of the emergency departments in a given region can be stored in a database or network server.

The term “navigation data” refers to programmatically determined data that identify and provide guidance information on the route to an emergency department. For example, the navigation data may provide route information for the medical transport, such as an ambulance, to travel to the nearest inpatient capable facility, outpatient only facility, or any other applicable emergency department.

The term “communication request” refers to an electronic request to establish one or more communication links between a local party and a remote party. In this regard, video audio signals and video image signals are transmitted/received between the local party and the remote party. Various networks may be used to facilitate the communications, including, for example, internet, Wi-Fi, LAN, WAN, cellular network, etc.

The term “network” may be home healthcare system for patient, network within patient's insurance coverage, or network designated by patient in advance. Information related to the patient network can be stored in a network database, and such information can be remotely accessed via the internet.

### Example System Architecture

Methods and apparatuses of the present invention may be embodied by any of a variety of devices. For example, methods and apparatuses of an example embodiment may be embodied by one or more networked devices, such as a server or other network entity, configured to communicate with other devices, such as one or more client devices. Additionally or alternatively, the computing device may include fixed computing devices, such as a personal computer or a computer workstation. Still further, example embodiments may be embodied by any of a variety of mobile devices, such as a portable digital assistant (PDA), mobile phone, smartphone, laptop computer, tablet computer, wearable device, or any combination of the aforementioned devices. These devices may be permanently or temporarily fixed (e.g., within a medical transport, such as an ambulance, ship, or helicopter), may be worn by a user, or may be portable or handheld.

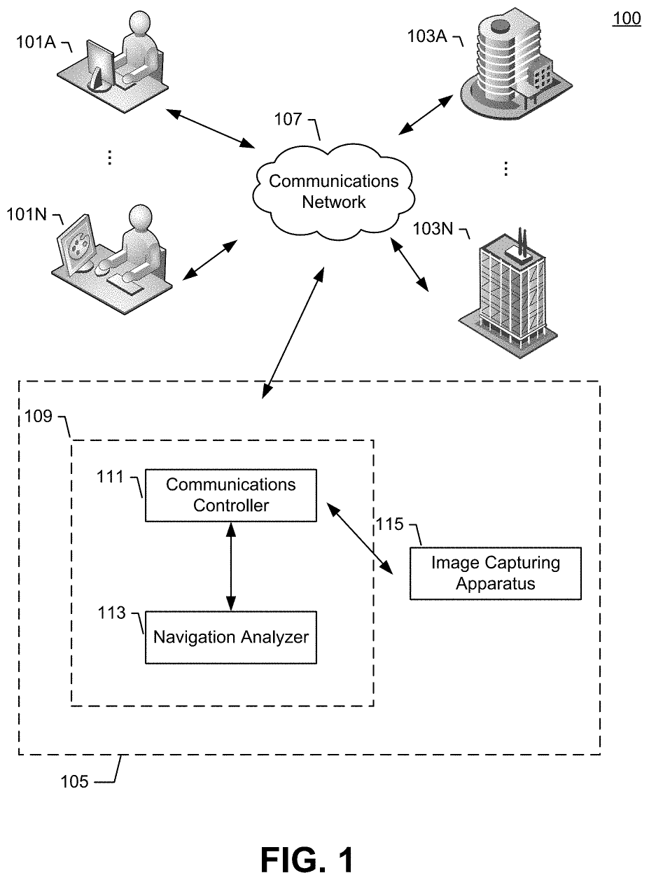

FIG. 1 illustrates an example overview diagram 100 illustrating how various embodiments of the present invention may operate. A medical transport telepresence system 105 may communicate with one or more computing devices operated by the remote parties 101A-101N via a communications network 107 (e.g., the Internet, Wi-Fi, LAN, WAN, cellular network, or the like). The medical transport telepresence system 105 may also communicate with one or more computing devices connected to the emergency departments 103A-103N via the communications network 107. In some embodiments, the remote parties 101A-101N may be located at one or more of the emergency departments 103A-103N.

The medical transport telepresence system 105 may comprise one or more image capturing apparatus 115 in communication with the computer system 109.

The computer system 109 may be embodied as a computer or computers as known in the art. The computer system 109 may provide for receiving of electronic data from various sources, including but not limited to the remote parties 101A-101N, emergency departments 103A-103N, and one or more image capturing apparatus 115. For example, the computer system 109 may be operable to receive and process diagnostic indications provided by the remote parties 101A-101N, and receive and process waiting time data from the one or more computing devices connected to the emergency departments 103A-103N. The computer system 109 may facilitate the generation of navigation data.

In some embodiments, the waiting time data may be stored in one or more remote servers (such as one or more database servers) and/or one or more computer storage devices (including floppy disks, USB flash drives, memory cards, memory sticks, tape cassettes, zip cassettes, computer hard drives, CDs and DVDs). In some embodiments, the waiting time data is historical waiting time data. In some embodiments, the waiting time data is forecasted based on the historical waiting time data. The computer system 109 receives and processes waiting time data from the one or more remote servers and/or other computer storage devices, which may or may not be within the emergency departments 103A-103N.

The remote parties 101A-101N may be connected to the communications network 107 using any computing device as defined above. Electronic data received by the medical transport telepresence system 105 from the remote parties 101A-101N may be provided in various forms and via various methods. In some preferred and non-limiting embodiments, one or more of such a computing device is a mobile device, such as a smart phone or tablet. The remote parties 101A-101N may execute an “app” to communicate with the medical transport telepresence system 105, including, for example, receiving diagnostic request, transmitting or receiving a communications request, and transmitting diagnostic indication. Such apps are typically designed to execute on mobile devices, such as tablets or smartphones. For example, an app may be provided that executes on mobile device operating systems such as Apple Inc.'s iOS®, Google Inc.'s Android®, or Microsoft Inc.'s Windows 10 Mobile®. These platforms typically provide frameworks that allow apps to communicate with one another and with particular hardware and software components of mobile devices. For example, the mobile operating systems named above each provide frameworks for interacting with location services circuitry, wired and wireless network interfaces, user contacts, and other applications. Communication with hardware and software modules executing outside of the app is typically provided via Application Programming Interfaces (APIs) provided by the mobile device operating system.

In some embodiments, the emergency departments 103A-103N may be connected to the communications network 107 using any computing device as defined above. In this regards, the emergency departments 103A-103N may transmit data associated with the emergency departments 103A-103N to the medical transport telepresence system 105 via the communications network 107 and/or receive data related to the subject from the medical transport telepresence system 105 and/or the remote parties 101A-101N (e.g., telehealth specialists). In some embodiments, the remote parties 101A-101N may be located at the emergency departments 103A-103N.

An example of a data flow for exchanging electronic information among various components is described below.

In some embodiments, the medical transport telepresence system 105 may use the one or more image capturing apparatus 115 to capture still and/or moving images of the subject in the medical transport. For example, two or more cameras may be mounted on the back (one on the left, one on the right) inside the medical transport to capture videos of the subject. In some embodiments, a single camera may be mounted in the medical transport. In some embodiments, the computer system 109 may have the image capturing apparatus 115 mounted thereto as an integral part. The image capturing apparatus 115 then transmits the captured still and/or moving image data to the communications controller 111 of the computer system 109.

In some embodiments, the present invention may use a gimbal to provide image stabilization. The gimbal may be a mechanical device that can sense and manipulate the orientation of the image capturing apparatus to correct for any movement of medical transport.

In some embodiments, the one or more image capturing apparatus 115 transmit the image data via one or more local wired networks, such as Ethernet. In some embodiments, the one or more image capturing apparatus 115 transmit the image data via one or more wireless networks, such as Bluetooth, ZigBee, etc. In some embodiments, the one or more image capturing apparatus 115 is an integral part of the computer system 109, and image data is transmitted via one or more circuitries. In some embodiments, the image data is transmitted based on one of the combinations of the above-mentioned method.

The computer system 109 then transmits still and/or moving images captured by the image capturing apparatus 115 to the one or more remote parties 101A-101N via the communications network 107, along with a diagnostic request including related data associated with the subject. For example, the computer system 109 may transmit the video captured by the image capturing apparatus 115 to the one or more remote parties 101A-101N, such as trained medical telehealth specialists, together with other data related to the subject, and request the trained medical telehealth specialists to provide diagnostic indications, such as whether the subject's symptoms are caused by large vessel occlusion, in the case of a cerebrovascular accident.

In some embodiments, communications controller 111 of the computer system 109 may also establish one or more communications links, which may include the video signals described above, with the one or more remote parties 101A-101N via multiple networks of the communications network 107. For example, the communications controller 111 may use a Wi-Fi network to transmit the video and/or audio signals to the one or more remote parties 101A-101N via the communications network 107 and, also, may use a separate cellular network to transmit the video and/or audio signals to the one or more remote parties 101A-101N. As another example, the communications controller 111 may use the Wi-Fi network to transmit the other data, including data related to the condition of the patient (e.g. vital signs such as body temperature, pulse rate, respiration rate, blood pressure, etc.).

Based on the communication links and data transmitted to the remote parties 101A-101N via the telepresence system, the one or more remote parties 101A-101N may then transmit diagnostic indications via the communications network 107 to the computer system 109 of the medical transport telepresence system 105. The diagnostic indications may be in the form of an electronic indication from the remote parties 101A-101N to the computer system 109. In some embodiments, the diagnostic indications may be an electronic indication over the audio link between the remote parties 101A-101N and the medical transport personnel (e.g., emergency medical technicians, etc.), which personnel then enter the indications into the computer system 109.

Various embodiments of the present invention provide technical improvements over existing, conventional systems. For example, the medical transport telepresence system 105 of the present invention provides stable communication with the remote party in real-time, thus enabling the medical transport personnel to conduct remote interaction with the telehealth specialist.

In some embodiments, the medical transport telepresence system 105 may also receive data from one or more computing devices connected to the one or more emergency departments 103A-103N regarding various estimated time measurements, such as door to needle time (“DTN”) interval and door to groin time (“DTG”) interval, of the one or more emergency departments 103A-103N. For example, a DTN time interval starts with a subject's arrival at the emergency department and ends with administration of tPA, including intermediate CT scanning and other diagnosis and waiting times. And the DTN time interval starts with a subject's arrival at an emergency department and ends with the start of a thrombectomy operation, including intermediate CT scanning and other diagnosis and waiting times. Such data is an important variable in software processing of optimal location designation, as described in details herein.

In some embodiments, waiting time data, average DTN, and/or DTG, in connection with each emergency department of a geographic area (e.g., the service area of a medical transport company) is stored locally on the medical transport telepresence system 105. In some embodiments, the waiting time data, average DTN, and/or DTG, is stored remotely on one or more remote servers as described above.

Upon receiving the diagnostic indications from the one or more remote parties 101A-101N and optionally receiving various time measurements from the one or more emergency departments 103A-103N, the computer system 109 utilizes the navigation analyzer 113 to generate navigation data, details of which are described further hereinafter.

### Example Apparatus for Implementing Embodiments of the Present Invention

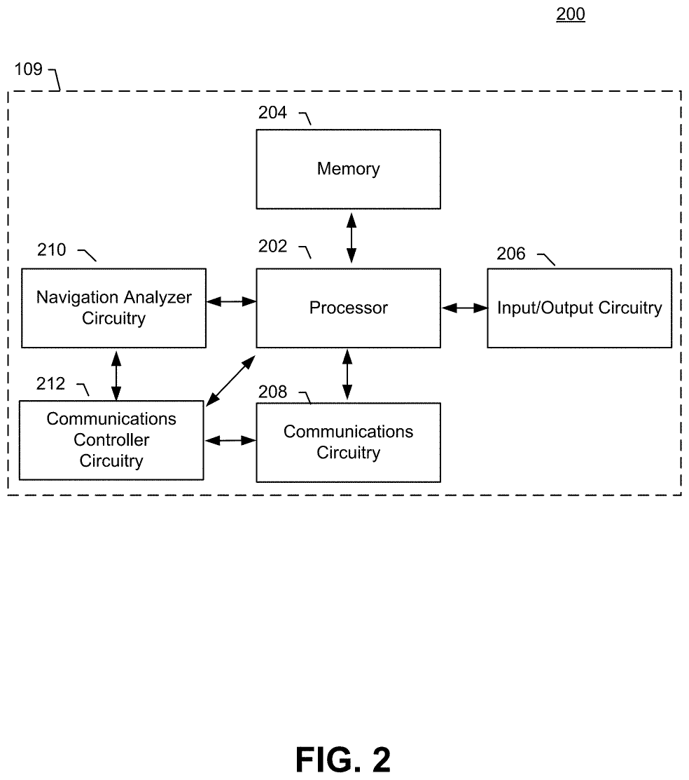

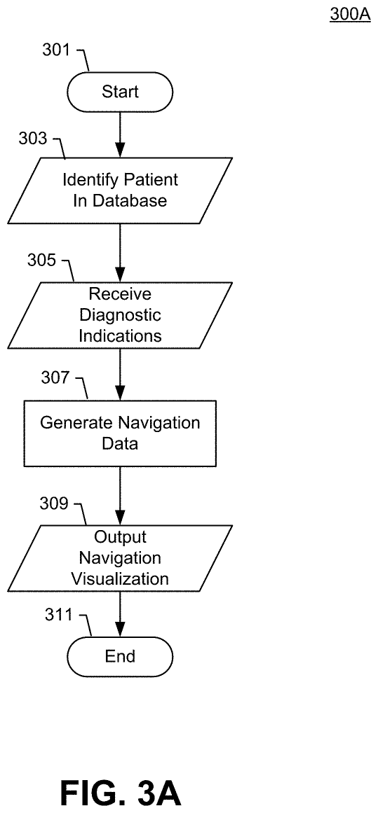

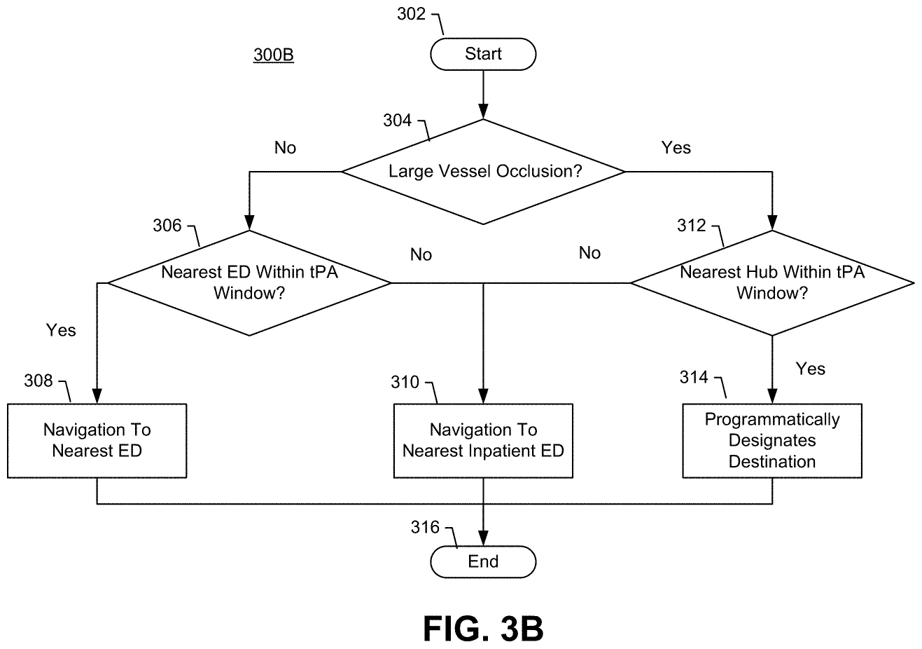

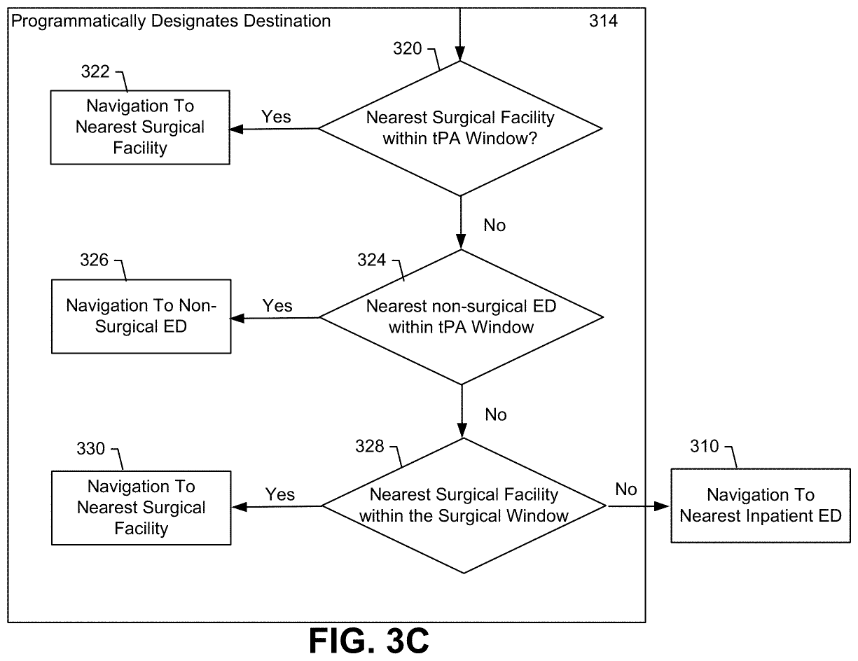

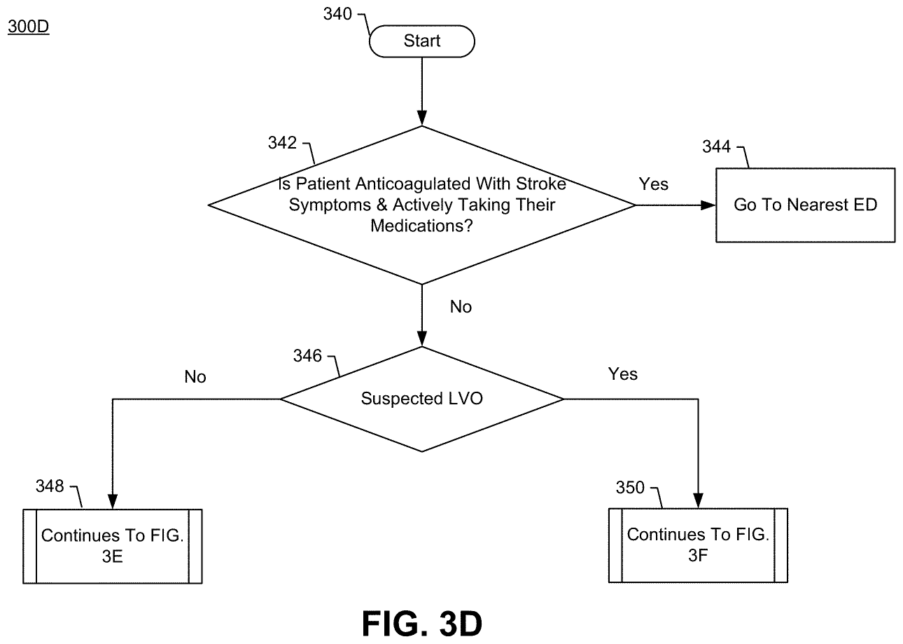

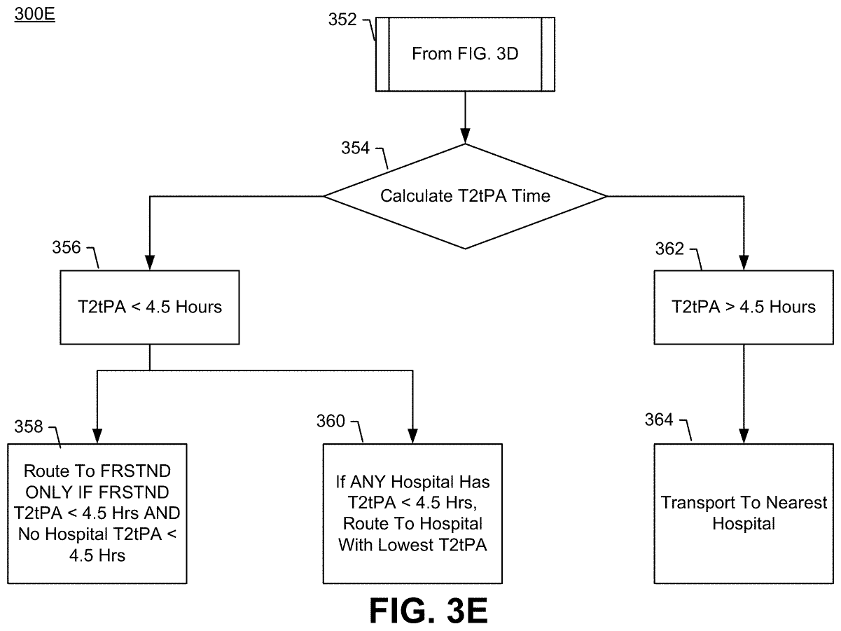

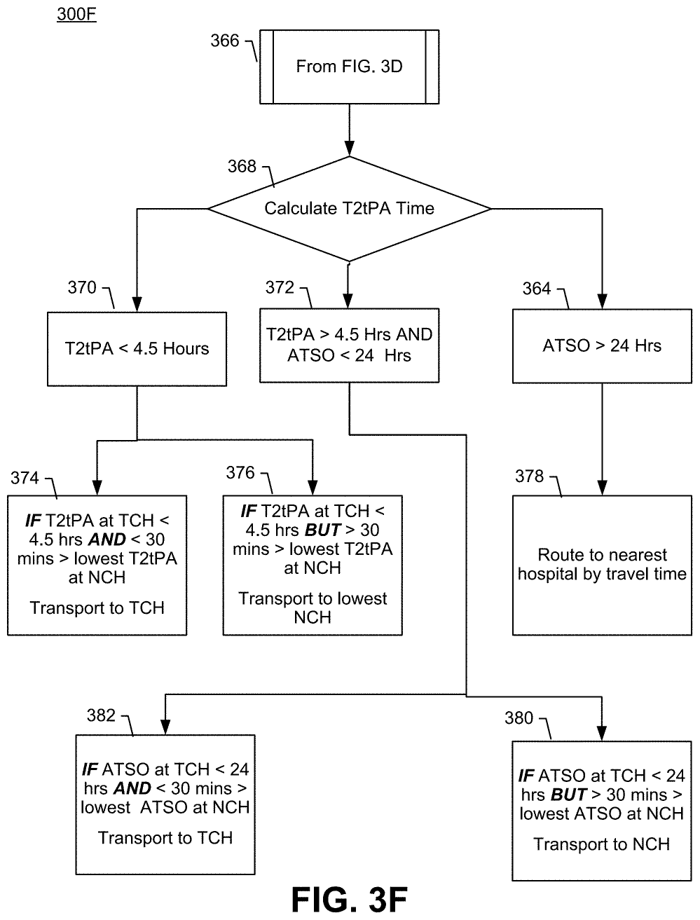

The computer system 109 may be embodied by one or more computing systems, such as apparatus 200 shown in FIG. 2. The apparatus 200 may include processor 202, memory 204, input/output circuitry 206, communications circuitry 208, navigation analyzer circuitry 210, and communications controller circuitry 212. The apparatus 200 may be configured to execute the operations described above with respect to FIG. 1 and below with respect to FIGS. 3A-5. Although these components 202-212 are described with respect to functional limitations, it should be understood that the particular implementations necessarily include the use of particular hardware. It should also be understood that certain of these components 202-212 may include similar or common hardware. For example, two sets of circuitry may both leverage use of the same processor, network interface, storage medium, or the like to perform their associated functions, such that duplicate hardware is not required for each set of circuitry.

The term “circuitry” should be understood broadly to include hardware and, in some embodiments, software for configuring the hardware. With respect to components of the apparatus, the term “circuitry” as used herein should therefore be understood to include particular hardware configured to perform the functions associated with the particular circuitry as described herein. For example, in some embodiments, “circuitry” may include processing circuitry, storage media, network interfaces, input/output devices, and the like. In some embodiments, other elements of the apparatus 200 may provide or supplement the functionality of particular circuitry. For example, the processor 202 may provide processing functionality, the memory 204 may provide storage functionality, the communications circuitry 208 may provide network interface functionality, and the like.

In some embodiments, the processor 202 (and/or co-processor or any other processing circuitry assisting or otherwise associated with the processor) may be in communication with the memory 204 via a bus for passing information among components of the apparatus 200. The memory 204 is non-transitory and may include, for example, one or more volatile and/or non-volatile memories. In other words, for example, the memory 204 may be an electronic storage device (e.g., a computer readable storage medium). The memory 204 may be configured to store information, data, content, applications, instructions, or the like, for enabling the apparatus to carry out various functions in accordance with example embodiments of the present invention.

The processor 202 may be embodied in a number of different ways and may, for example, include one or more processing devices configured to perform independently. In some preferred and non-limiting embodiments, the processor 202 may include one or more processors configured in tandem via a bus to enable independent execution of instructions, pipelining, and/or multithreading. The use of the term “processing circuitry” may be understood to include a single core processor, a multi-core processor, multiple processors internal to the apparatus, and/or remote or “cloud” processors.

In some preferred and non-limiting embodiments, the processor 202 may be configured to execute instructions stored in the memory 204 or otherwise accessible to the processor 202. In some preferred and non-limiting embodiments, the processor 202 may be configured to execute hard-coded functionalities. As such, whether configured by hardware or software methods, or by a combination thereof, the processor 202 may represent an entity (e.g., physically embodied in circuitry) capable of performing operations according to an embodiment of the present invention while configured accordingly. Alternatively, as another example, when the processor 202 is embodied as an executor of software instructions, the instructions may specifically configure the processor to perform the algorithms and/or operations described herein when the instructions are executed.

In some embodiments, the apparatus 200 may include input/output circuitry 206 that may, in turn, be in communication with processor 202 to provide output to the user and, in some embodiments, to receive an indication of a user input. The input/output circuitry 206 may comprise a user interface and may include a display, and may comprise a web user interface, a mobile application, a client device, a kiosk, or the like. In some embodiments, the input/output circuitry 206 may also include a keyboard, a mouse, a joystick, a touch screen, touch areas, soft keys, a microphone, a speaker, or other input/output mechanisms. The processor and/or user interface circuitry comprising the processor may be configured to control one or more functions of one or more user interface elements through computer program instructions (e.g., software and/or firmware) stored on a memory accessible to the processor (e.g., memory 204, and/or the like).

In some embodiments, the input/output circuitry 206 further includes circuitries that are configured to receive and transmit video/audio signals. For example, the input/output circuitry may include a camera, a microphone, and/or other video capturing circuitry. The input/output circuitry may further include a speaker, a display, and/or other video display circuitry. In some embodiments, these circuitries are separated from the apparatus 200.

The communications circuitry 208 may be any means such as a device or circuitry embodied in either hardware or a combination of hardware and software that is configured to receive and/or transmit data from/to a network and/or any other device, circuitry, or module in communication with the apparatus 200. In this regard, the communications circuitry 208 may include, for example, a network interface for enabling communications with a wired or wireless communication network. For example, the communications circuitry 208 may include one or more network interface cards, antennae, buses, switches, routers, modems, and supporting hardware and/or software, or any other device suitable for enabling communications via a network. Additionally or alternatively, the communications circuitry 208 may include the circuitry for interacting with the antenna/antennae to cause transmission of signals via the antenna/antennae or to handle receipt of signals received via the antenna/antennae.

Navigation analyzer circuitry 210 includes hardware configured to determine and identify a target or recommended emergency department, and may further generate or receive navigation data. In some embodiments, the analyzer circuitry 210 is configured to receive at least one of the first location of the medical transport and a current location of the medical transport, receive the diagnostic indication, generate navigation data comprising navigation instructions from the at least one of the first location of the medical transport and the current location of the medical transport to an emergency department based on the diagnostic indication, and transmit the navigation data to the at least one display in the medical transport.

For example, the analyzer circuitry 210 may determine a current location of the medical transport at location X. Upon receiving the diagnostic indication, the analyzer circuitry 210 may analyze the diagnostic indication and determine an emergency department Y as the destination (details of which are described hereinafter). The analyzer circuitry 210 retrieves the location of the emergency department Y, and generates navigation data, which includes navigation instructions from location X to the emergency department Y. The analyzer circuitry 210 transmits the navigation data to the at least one display in the medical transport.

In some embodiments, the analyzer circuitry 210 may comprise one or more additional circuitries to generate navigation data. Continuing from the previous example, after determining the emergency department Y as the destination and retrieving the location of the emergency department Y, a portion of the analyzer circuitry 210 may transmit the location information to another portion of the analyzer circuitry 210 via an Application Programming Interface (API). The circuitry may provide web mapping service, which includes generating navigation data and transmitting the navigation data to the local analyzer circuitry. Upon receiving the navigation data, the local analyzer circuitry transmits the navigation data to the at least one display in the medical transport.

The navigation analyzer circuitry 210 may utilize processing circuitry, such as the processor 202, to perform these actions. The navigation analyzer circuitry 210 may receive data from the communications controller circuitry 212. In some embodiments, the navigation analyzer circuitry 210 may include or be connected to a global positioning system (GPS) antenna, or the like, for receiving positioning information. As described above, the navigation analyzer circuitry 210 may include one or more sub-circuitries for performing various portions of the functionality of the navigation analyzer.

In some embodiments, the navigation analyzer circuitry 210 may include a separate processor, specially configured Field Programmable Gate Array (FPGA), or Application Specific Integrated Circuit (ASIC). In some implementations, the communications controller circuitry 212, described below, may be sub-circuitries belonging to navigation analyzer circuitry 210. The navigation analyzer circuitry 210 may be implemented using hardware components of the apparatus configured by either hardware or software for implementing the functions described herein.

The communications controller circuitry 212 includes hardware configured to control communications between the computer system 109 and other systems, including the communications between the medical transport telepresence system 105 and the one or more remote parties 101A-101N. Further, the communications controller circuitry 212 includes hardware configured to control communications between computer system 109 and other components of the medical transport telepresence system, including, for example, the image capturing apparatus 115, one or more sensors that generate data related to the conditions of the patient (e.g., vital signs monitors), etc. The communications controller circuitry 212 may utilize low energy Bluetooth, ZigBee, Wi-Fi, near-field communication (NFC), radio frequency (Rf), IR and/or any combination of the communication protocols, or other applicable communication protocols.

In some embodiments, the communications controller circuitry 212 may utilize processing circuitry, such as the processor 202, to perform these actions. However, it should also be appreciated that, in some embodiments, the communications controller circuitry 212 may include a separate processor, specially configured Field Programmable Gate Array (FPGA), or Application Specific Integrated Circuit (ASIC) for performing the functions described herein. The communications controller circuitry 212 may be implemented using hardware components of the apparatus configured by either hardware or software for implementing these planned functions.

As will be appreciated, any such computer program instructions and/or other type of code may be loaded onto a computer, processor or other programmable apparatus's circuitry to produce a machine, such that the computer, processor or other programmable circuitry that execute the code on the machine creates the means for implementing various functions, including those described herein.

It is also noted that all or some of the information discussed herein can be based on data that is received, generated and/or maintained by one or more components of apparatus 200. In some embodiments, one or more external systems (such as a remote cloud computing and/or data storage system) may also be leveraged to provide at least some of the functionality discussed herein.

As described above and as will be appreciated based on this disclosure, embodiments of the present invention may be configured as methods, mobile devices, medical transports, medical transport telepresence systems, backend network devices, and the like. Accordingly, embodiments may comprise various means including entirely of hardware or any combination of hardware and software. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices.

### Example Processes of Embodiments of the Present Invention

Embodiments of the present invention achieve the goals of improving the identification of a subject and the diagnosis of a subject and generating related navigation data, which allows subjects to be efficiently routed such that they efficiently receive the appropriate treatment at an optimal location. Various processes described herein achieve these goals and overcome various technical difficulties.

### Navigation Data Generation

FIGS. 3A-3F illustrate example processes 300A, 300B, 300D, 300E, and 300F including data flows between the computer system 109, the one or more remote parties 101A-101N, and other components in generating navigation data in accordance with some embodiments of the present invention. As discussed herein, each of the time windows and navigation times may include the respective wait times (DTN and DTG) and other transactional times associated with the patient receiving a given treatment when determining which facility to recommend. For example, an emergency department that is a 15 minute drive away and has a 60 minute DTN time may be selected over an emergency department that is a 5 minute drive away but has a 90 minute DTN time when searching for a tPA administrable facility.

The process 300A starts at block 301. In some embodiments, the present invention may receive an indication, such as a phone call and/or video call, that a subject is in need of treatment.

At block 303, the computer system 109 may receive one or more inputs related to the conditions of the subject. For example, the computer system 109 may identify information related to the subject in a database, as described in details hereinafter. The computer system 109 may also receive a code identification of the subject indicating that the subject is having a cerebrovascular accident. Other inputs may include the last known well time of the subject, a programmatically calculated stroke score, patient identification data, patient insurance data, patient accountable care organization data, and patient medical history data. Further, additional information, such as whether medical consent has been received and other testing information may be received and updated during process 300A. These inputs may be manually entered by the medical transport personnel (e.g., EMT) or received from a networked computing system (e.g., via a wearable medical ID tag or from a central medical server after inputting basic information about the subject). After receiving related data, the computer system 109 may then transmit all or some of these data, together with a diagnostic request, to the one or more remote parties 101A-101N. In another embodiment, the telehealth specialist may enter the data described above based on information conveyed to them. In some embodiments, the diagnostic request may only be triggered in certain situations (e.g., a request for neurological diagnosis when a probable cerebrovascular accident is detected by the medical transport personnel).

At block 305, the computer system 109 receives diagnostic indications from the one or more remote parties 101A-101N. The indications may be based on vital signs transmitted from the computer system 109, visual diagnosis of the subject by the remote party over the telepresence system 105, and/or feedback from the emergency medical personnel. The diagnostic indication may include data indicating the diagnosis of the subject. For example, the diagnostic indications may indicate that the subject is having a cerebrovascular accident due to large vessel occlusion. The diagnostic indications may also include other information such as emergency treatments that need to be administered on the subject in the medical transport.

In various embodiments, the system may use grading systems to determine whether the patient is likely to experience a stroke. For example, the NIHSS or FENG-D scales can be used to determine the severity of the stroke. In various embodiments, the system's grading scale may be based on the clinical judgment of a remote party.

At block 307, the computer system 109 generates navigation data based on the received diagnostic indications. The navigation data may include at least a determination of a recommended emergency room to which the subject should be taken based on the aforementioned input data, and the navigation data may include turn-by-turn instructions to reach the recommended emergency room. In some embodiments, the navigation data may be generated in accordance with the process as illustrated in FIG. 3B.

At block 309, the computer system 109 outputs the determination of the recommended emergency room, which may also include a visualization of the navigation data. For example, the computer system 109 may display an electronic map and associated direction information on a computer screen for guiding the medical transport to the destination as programmatically determined by the computer system 109 at block 307. In some embodiments, the output may only be a textual indication of the recommended emergency room.

The process 300A ends at block 311.

Referring now to FIG. 3B, an example process 300B for generating navigation data is shown.

The process 300B starts at block 302 at a point where a probable cerebrovascular accident has been identified by the medical transport personnel using standard symptom identification techniques. At block 304, the computer system 109 determines whether the received diagnostic indication includes data indicating that the condition of the subject is caused by large vessel occlusion. In some embodiments, the diagnostic indication is in the form of video or audio signal (i.e. the telehealth specialist verbally indicates his or her diagnosis via the medical transport telepresence system).

If the computer system 109 determines that the diagnostic indication does not include such data or instead indicates a non-LVO, the process 300B continues to block 306, where the computer system 109 determines whether the medical transport is able to deliver the subject to the nearest tPA-administrable emergency department, which may include any inpatient capable facility or outpatient only facility, within the time window to administer tPA, which may include the DTN time. If yes, then the process 300B continues to block 308, where the computer system 109 generates navigation data directing the medical transport to the nearest emergency room. In some embodiments, no restriction may be placed on the type of tPA-administrable emergency department that is used. For example, if the process 300B determines that a neurosurgical facility is also the nearest tPA-administrable emergency department, the computer system 109 may direct a subject in block 308 to the neurosurgical facility because it is nearest.

If, at block 306, the computer system 109 determines that the medical transport is unable to arrive at the nearest outpatient only facility within a certain time window to administer tPA, then the process 300B continues to block 310, where the computer system 109 generates navigation data directing the medical transport to the nearest inpatient capable facility. In some embodiments, the process 300B utilizes standard navigation application programming interface (API) to generate navigation data to the determined emergency department once the determined emergency department is identified by the algorithm.

At block 304, if the computer system 109 determines that the diagnostic indication includes data indicating that the condition of the subject is caused by large vessel occlusion, then the process 300B continues to block 312, where the computer system 109 may determine whether the medical transport is able to deliver the subject to the nearest emergency department within the tPA window and may determine if the subject is a surgical candidate for thrombectomy. Determination that the subject is a surgical candidate may include determining that the patient can reach a neurosurgical emergency department within a predetermined window (e.g., 6 hours or 24 hours for certain thrombectomies), and/or that the patient is physically capable of enduring a surgery. In some embodiments, the diagnostic indication is in the form of verbal communication from the telehealth specialist via the medical transport telepresence system regarding the condition of the patient.

If yes, then the process 300B continues to block 314, where the computer system 109 generates navigation data directing the medical transport to the programmatically designated destination. With reference to FIG. 3C, an example embodiment of the programmatic determination of block 314 is shown. In the depicted embodiment, the system may prioritize administering tPA wherever possible. In such embodiments, the process at block 320 may determine if the subject can reach the nearest surgical facility (e.g., a facility capable of both performing thrombectomies and administering tPA) within the tPA window. If yes, the process may proceed to block 322 and direct the subject to the identified nearest surgical facility.

If the subject cannot reach the nearest surgical emergency department within the tPA window, the process may proceed to block 324 and determine if the subject can reach the nearest non-surgical emergency department within the tPA window. As described herein, a “non-surgical emergency department” may still be capable of performing some surgery, just not the surgery required by the subject. If yes, the process may proceed to block 326 and direct the subject to the identified nearest non-surgical facility.

If the subject cannot reach the nearest non-surgical emergency department, the process may proceed to block 328 and determine if the subject can reach the nearest surgical emergency department within a surgical window, which may be greater than the tPA window. If yes, the process may proceed to block 330 and direct the subject to the identified nearest surgical facility. If no, the process may exit to block 310 (also shown on FIG. 3B) and navigate the patient to the nearest inpatient capable emergency department, regardless of tPA or surgical capability.

If, at block 312, the computer system 109 determines that the medical transport is unable to arrive at the nearest tPA-administrable facility within the time window to administer tPA and the patient is not a surgical candidate, then the process 300B continues to block 310, where the computer system 109 generates navigation data directing the medical transport to the nearest inpatient capable facility.

The process 300B ends at block 316.

In some embodiments, determining transport times in the process 300B first determines the current location of the medical transport in relation to the locations of emergency departments in the area. The process 300B then calculates the drive time to each of these emergency departments, and determines the DTN and/or DTG time for each of them. The process 300B further determines the closest emergency department in a required category (e.g. any facility, inpatient capable facility, tPA administrable facility, surgical facility, etc.). In some further embodiments, the process may determine the closest emergency department in all categories simultaneously. Thus, where tPA is needed but thrombectomy is not (e.g., non-LVO), the system may direct the subject to the nearest tPA-administrable facility if one is in range. If tPA cannot be administered because no tPA-administrable facility is in range of the cutoff time (e.g., 4.5 hours from last known well time), the system may direct the subject to the nearest inpatient capable emergency department, regardless of tPA capability. If surgery is needed (e.g., LVO), the system may determine the desired destination using the algorithm discussed above, which may prioritize tPA, surgery, or weighting of factors, including these two and others, such as patient specific information.

In some embodiments, the process 300B may further determine the recommended emergency department based on the patient specific information, such as the patient's medical history, the patient's health insurance, the patient's ACO, or a hospital's historical treatment of similarly diagnosed patients. For example, the process 300B may determine locations of health care providers that are in the patient's insurer's network, and prioritize these health care providers over others that are not in the patient's insurer's network. In some embodiments, the subject's treatment is always prioritized over the hospital or network preference (e.g., if an in-network hospital is not in range, the system will automatically select an out-of-network hospital that may provide treatment).

Referring now to FIG. 3D, an example process 300D in accordance with various embodiments of the present invention is shown.

The process 300D starts at block 340. At Block 342, the process 300D determines whether the patient anticoagulated with stroke symptoms and actively taking their medications. If yes, the process 300D proceeds to generating navigation data comprising navigation instructions to the nearest emergency department at block 344. In some embodiments, the nearest emergency department is a Freestanding Emergency Room (“FRSTND”), which does not have the ability to admit patients for overnight stay. In some embodiments, the nearest emergency department is an inpatient hospital lacking the ability to perform thrombectomy (“NCH”). In some embodiments, the nearest emergency department is a Thrombectomy capable hospital (“TCH”). In some embodiments, possible bleed strokes go to the nearest ER to stabilize from blood thinners.

If, at block 342, the process 300D determines that the patient is not anticoagulated with stroke symptoms and. ro not actively taking their medications, the processes 300D process to block 346, where the process 300D determines whether the patient has suspected LVO. If yes, then the processes 300D continues to FIG. 3G at block 350. If no, then the process 300D proceeds to FIG. 3F at block 348.

FIG. 3E illustrates a process 300E for patient deemed not having a LVO, starting at block 352. At block 354, the process 300E calculates the Total Time to tPA (“T2tPA”) times based on the following formula:

Total Time to tPA (T2tPA)=Travel time+Elapsed time+Site averag tPA time

If the T2tPA time is less than 4.5 hours (as shown in block 362), the process 300E proceeds to block 364, which generates navigation data comprising navigation instructions to the nearest hospital. Here, the process 300E may further display the Arrival Time Since Onset (“ATSO”) to the stroke team at the nearest hospital, which can be calculated based on the following formula:

Arrival Time Since Onset (ATSO)=Elapsed time+Travel time

If the T2tPA time is more than 4.5 hours (as shown in block 356), the process 300E may generate navigation data comprising navigation instructions to the Freestanding Emergency Room (“FRSTND”) only if the T2tPA time to the FRSTND is less than 4.5 hours, and no other hospital has a T2tPA time more than 4.5 hours, as shown in block 358. If any hospital has a T2tPA time less than 4.5 hours (as shown in block 360), the process 300E may generate navigation data comprising navigation instructions to a hospital with the lowest T2tPA time.

FIG. 3F illustrates a process 300F for patient deemed likely having a LVO. The process 300F starts at block 366. At block 368, the process 300F calculates the T2tPA and the ATSO times. If process 300F determines that the ATSO time exceeds 24 hours (as shown in block 364), the process 300F generates navigation data comprising navigation instructions to the nearest hospital by travel time (as shown in block 378).

If the process 300F determines that the T2tPA time is more than 4.5 hours and the ATSO is less than 24 hours (as shown in block 372), the process 300F determines if the ATSO time at the nearest Thrombectomy Capable Hospital (“TCH”) is less than 24 hours and less than 30 minutes plus the lowest ATSO at an inpatient hospital lacking the ability to perform thrombectomy (“NCH”). If yes, then the process 300F generates navigation data comprising navigation instructions to the TCH, as shown in block 382. If the ATSO time at the nearest TCH is less than 24 hours but more than 30 minutes plus the lowest ATSO at the NCH, the process 300F generates navigation data comprising navigation instructions to the NCH, as shown in block 380.

If the process 300F determines that the T2tPA time is less than 4.5 hours (as shown in block 370), the process 300F determines if the T2tPA time at the nearest Thrombectomy Capable Hospital (“TCH”) is less than 4.5 hours and less than 30 minutes plus the lowest T2tPA at an inpatient hospital lacking the ability to perform thrombectomy (“NCH”). If yes, then the process 300F generates navigation data comprising navigation instructions to the TCH, as shown in block 374. If the T2tPA time at the nearest TCH is less than 4.5 hours but more than 30 minutes plus the lowest T2tPA at the NCH, the process 300F generates navigation data comprising navigation instructions to the NCH, as shown in block 376.

### Subject Diagnosis

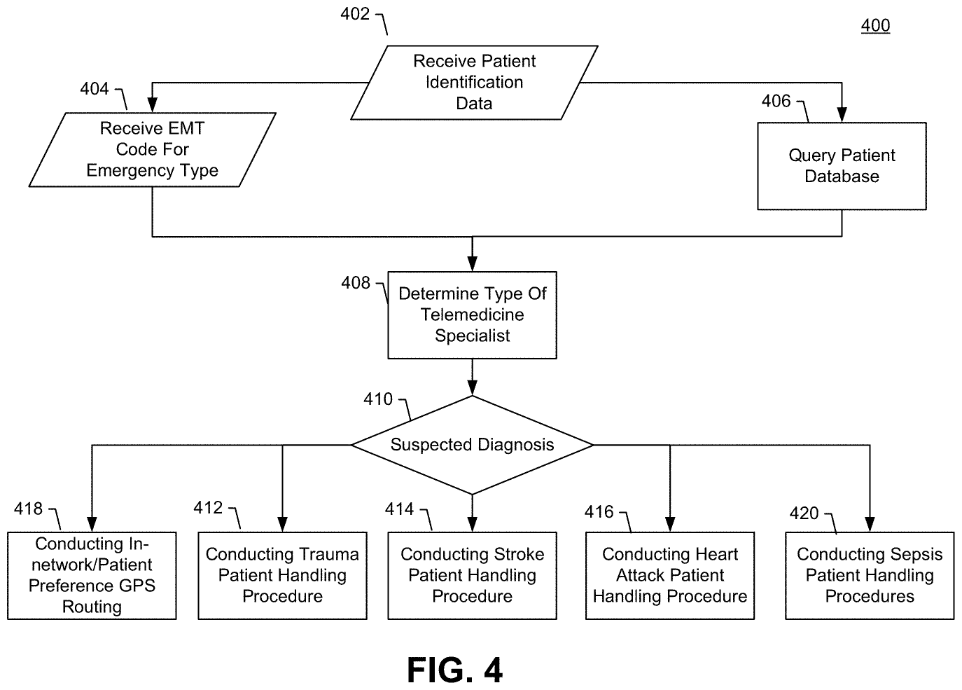

FIG. 4 illustrates an example process 400 including data flows in diagnosing a subject in need of treatment in accordance with some embodiments of the present invention. The process 400 may be carried out by a computer apparatus, such as the computer system 109 described above with regard to FIGS. 1 and 2.

Referring now to block 402, the computer apparatus, such as computer system 109, may receive patient identification data. Such data may be determined based on, for example, a medical bracelet that the patient wears. In some embodiments, the medical bracelet may contain patient specific information, such as physical information regarding the patient, medical history, next of kin, healthcare power of attorney, medications the patient is taking, patient's insurance, patient's insurer network, doctor preferences, hospital preference, and/or other information the patient chooses to make available. Such information, in some embodiments, may be included on the bracelet using biometric data, QR codes or may be encoded to communicate information wireless, such as via Bluetooth, radio frequency identification (RFID), or near field communication (NFC). In some embodiments, the medical transport personnel may enter the data into the medical transport telepresence system 105 manually from a conscious subject, a relative, or the subject's identification papers.

Upon receiving the patient identification data, including the identity of the patient, the computer apparatus, such as computer system 109, may query a patient database for information related to the patient (as shown in block 406). For example, the computer system 109 may identify patient logged history, patient's insurance information, accountable care organization (ACO) information of the patient, home hospital of the patient, and other pertinent medical and personal history of the patient. The computer apparatus, such as computer system 109, may further access data such as advance directives of the patient. Each of the pieces of data detailed herein may be used individually or collectively in determining the navigation data including the recommended emergency department.

The computer apparatus, such as computer system 109, may receive a code from an emergency medical technician indicating the emergency type of the patient at block 404. For example, a code “A” may indicate that the patient may be suffering from a trauma; a code “B” may indicate that the patient may have a cerebrovascular accident; a code “C” may indicate that the patient is having a heart attack or ST-segment elevation myocardial infarction; a code “D” may indicate that the patient is unstable; a code “E” may indicate that the above-mentioned codes are not applicable to the patient's symptoms.

At block 408, the computer apparatus, such as computer system 109, may select a telehealth specialist from a pool of one or more candidates and transmit relevant information to the telehealth specialist. At block 410, the computer apparatus, such as computer system 109, may request a diagnosis from the telehealth specialist.

If the computer apparatus, such as computer system 109, receives a diagnosis that the patient has a trauma, the process 400 proceeds to block 412, where the computer apparatus, such as computer system 109, generates navigation data based on the trauma type, GPS location, and whether the trauma has been stabilized.

If the computer apparatus, such as computer system 109, receives a diagnosis that the patient is having a cerebrovascular accident, the process 400 proceeds to block 414, where the computer apparatus, such as computer system 109 as described with regards to FIGS. 1 and 2, generates navigation data based on, for example, whether the patient is having large vessel occlusion as described with reference to FIGS. 3A and 3B. The navigation data may include a recommended emergency department based on the algorithms discussed herein and may include turn-by-turn instructions from the current location of the subject (and medical transport) to the indicated emergency department.

If the computer apparatus, such as computer system 109, receives a diagnosis that the patient is having a heart attack, the process 400 proceeds to block 416, where the computer apparatus, such as computer system 109, transmits information, such as electrocardiography and/or patient information, to the destination on-call cardiology department. The computer apparatus generates navigation data based on the suspected diagnosis, such as a heart attack, and capabilities of nearby emergency rooms and treatment facilities. The navigation data may include a recommended emergency department based on the diagnosis, and the navigation data may include turn-by-turn instructions from the current location of the subject (and medical transport) to the indicated emergency department.

Further, as shown in block 418, the process 400 may generate GPS routing information based on determining whether the location is in-network or based on patient preference. As shown in block 420, the process 400 may conduct sepsis patient handling procedures.

### Telepresence Communications

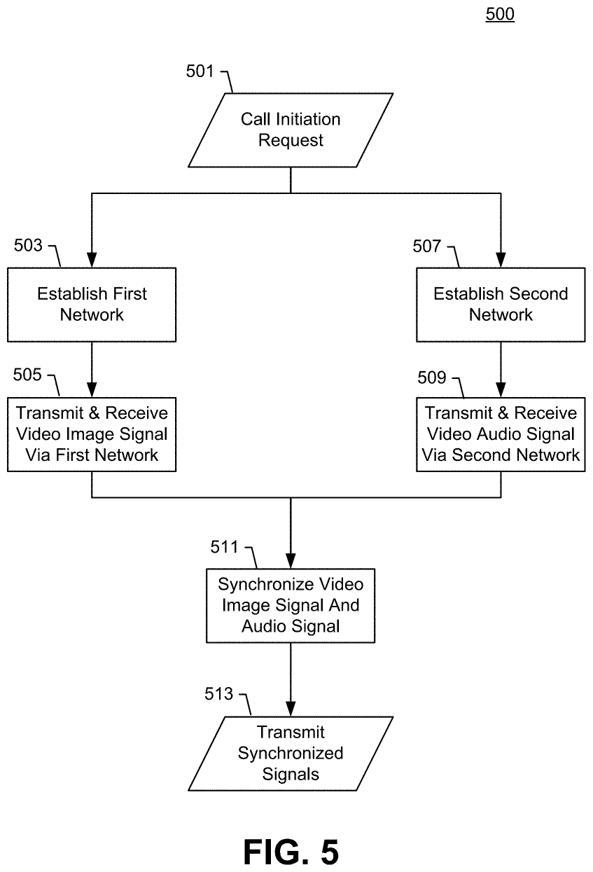

In some embodiments, the diagnostic indication may be generated based on data and clinical examinations performed and received through the telepresence system. FIG. 5 illustrates an example process 500 including data flows in establishing one or more communication links with one or more remote parties 101A-101N in accordance with some embodiments of the present invention. These communication links are encrypted and satisfy various requirements, including HIPAA. In some embodiments this encryption may be AES 256 bit encryption.

The process 500 begins at block 501, where a computer apparatus, such as computer system 109 as described with regard to FIGS. 1 and 2, may receive a request to initiate a call, such as a video call, audio call, or both. The request may come from either the subject-side computer (e.g., a computer system 109 in the medical transport) or from a doctor or telehealth specialist-side device, either of which may be at an emergency department.

Upon receiving the call initiation request, the process 500 proceeds to block 503, where the computer apparatus, such as computer system 109, may establish a first communication link via a first network (either an existing or newly established network) and transmits, for example, a video image signal via the first communication link at block 505. For example, with references to FIG. 1, upon receiving the call initiation request, the process 500 may establish a communication link between the medical transport telepresence system 105 and the remote parties 101A-101N to transmit the video image signal via a Wi-Fi network connected to the communications network 107.

Upon receiving the call initiation request, the process 500 may also proceed to block 507, where the computer apparatus, such as computer system 109, establishes a second communication link via a second network (either an existing or newly established network) and transmits, for example, an audio signal via the second network at block 509. For example, with references to FIG. 1, upon receiving the call initiation request, the process 500 may establish a communication link between the medical transport telepresence system 105 and the remote parties 101A-101N to transmit the video image signal via a cellular network connected to the communications network 107.

In some embodiments, both the first network and second network may be wireless networks. In some embodiments, the first network is separated from the second network. Such separate networks allow for redundancy in case one network were to fail, which may result in a dropped call.

At block 511, the computer apparatus, such as computer system 109, synchronizes video image signal and audio signal. At block 513, the computer apparatus, such as computer system 109, proceeds to transmit the video image signal and the video audio signal via a communications network, such as the communications network 107 as described in connection with FIG. 1.

In some embodiments, the computer apparatus, such as computer system 109, is also configured to receive a video image signal via the first network and an audio signal via the second network. The computer apparatus, such as computer system 109, then outputs the received video image signal and the received audio signal.

In some embodiments, a video call between the medical transport and telehealth specialist may transmit the video from the computer over a Wi-Fi signal via a Wi-Fi adapter and may transmit the audio over a cellular wireless signal via a cellular adapter. In such embodiments, the cellular signal may continue to transmit audio if the Wi-Fi loses signal or has insufficient bandwidth to enable a more robust connection and prevent dropped calls with the medical transport. In some embodiments, the Wi-Fi may carry both an audio and video signal. In such embodiments, an additional cellular audio signal may be transmitted while the Wi-Fi audio signal is disabled. The medical transport may broadcast one or both of the networks (e.g., an onboard Wi-Fi network), which may receive its signal via a cellular connection. In such embodiments, the ultimate connection for the video and audio signals may be two separate cellular network connections (e.g., one cellular connection via the Wi-Fi and one direct to the telepresence system).

### Example Device for Implementing Embodiments of the Present Invention

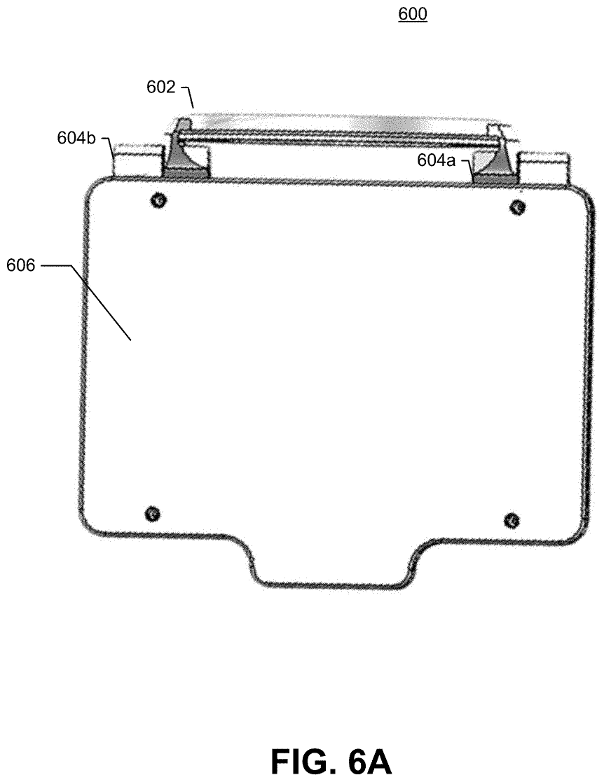

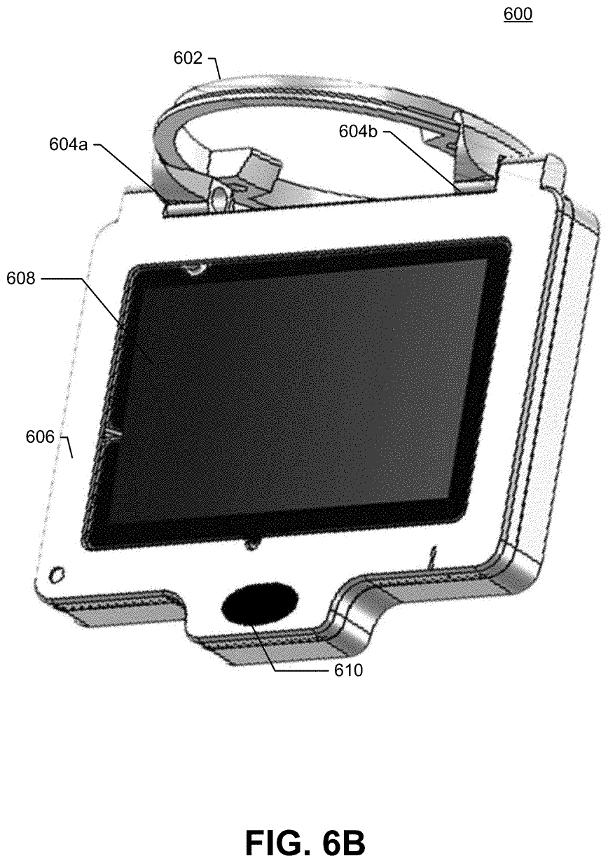

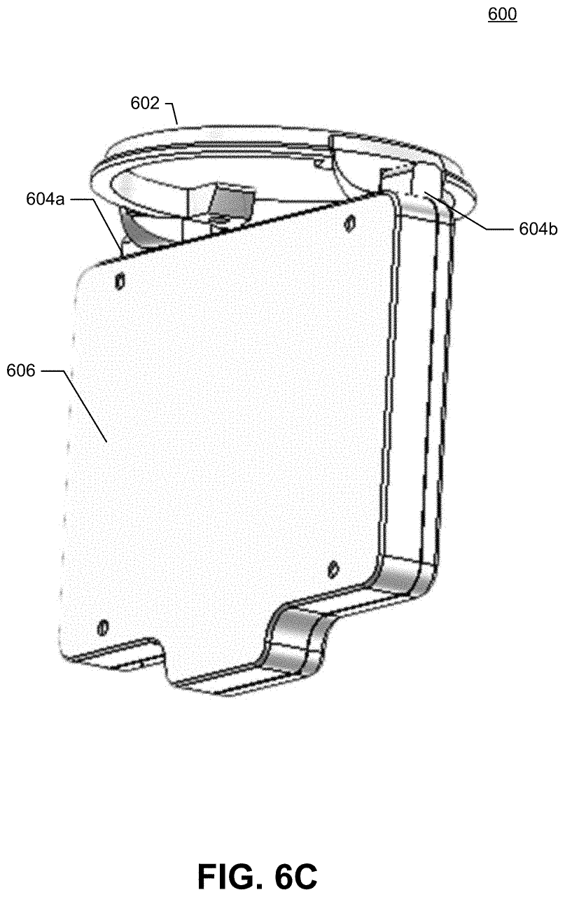

In some embodiments, the computer system 109 may be embodied by one or more computing devices, and may be mounted to a medical transport as part of an apparatus 600 (e.g., a tablet device) as shown in FIGS. 6A-6C. In some embodiments, the apparatus 600 shown in FIGS. 6A-6C may include the display and may include the camera for the computer system 109. In some embodiments, some or all of the computer system may be integral with the apparatus or some or all of the computer system may be mounted elsewhere in the medical transport.

Referring now to FIG. 6A, a back view of the example apparatus 600 is shown. The apparatus 600 may have a mount 602 that connects the apparatus 600 to an interior wall or ceiling of the medical transport. In some embodiments, the mount 602 is a ring mount that allows the apparatus 600 to rotate relative to the medical transport. In some embodiments, the mount 602 has one or more arms (not shown) connecting the apparatus 600 to the medical transport while allowing the apparatus 600 to be extended and articulated as needed.

The mount 602 is connected to the housing 606 via two or more hinges 604a, 604b. The hinges 604a, 604b enable the housing 606 to be rotated about an axis. With reference to FIGS. 6A-6C, the hinges 604a, 604b may include a lip that slidingly engages a corresponding flange on the mount 602 to allow the apparatus 600 to rotate circularly about the axis (e.g., an axis perpendicular to the plane of the mount 602 passing through the center of the mount), while preventing the apparatus 600 from falling or detaching from the mount. The hinges 604a, 604b may include a second axis of rotation to allow the apparatus 600 to fold substantially flat against the wall or ceiling of the medical transport. To fold the apparatus 600, the hinges 604a, 604b may pivot about an axis that extends between the hinges (e.g., an axis parallel to the plane of the mount 602 passing through both hinges 604a, 604b). For example, before the medical transport picks up a subject, the apparatus 600 may be in a “closed” position where the housing 606 touches or is disposed proximate an interior wall or ceiling of the medical transport (e.g., if an ambulance is used, the apparatus 600 is rotated about the second axis approximately 90 degrees from the position shown in FIGS. 6A-6C). After the medical transport picks up the subject, the housing 606 may be pivoted perpendicular to the interior wall or ceiling of the medical transport (an “open” position).

Referring now to FIGS. 6B and 6C, a front perspective view and a rear perspective view of the apparatus 600 are shown. As illustrated in FIG. 6B, the housing 606 may provide space for a tablet computer (or standalone display) 608. In some embodiments, the computer system 109, as described above with reference to FIGS. 1 and 2, may be embodied in the tablet computer 608. The tablet computer 608 may also include a camera and/or a microphone. The housing 606 may further include a button 610, which enables a user to start a video call utilizing the camera and the microphone of the tablet computer 608 by pressing the button 610. The camera is configured to generate video signals, and the microphone is configured to generate audio signals. In some embodiments, the camera, microphone, and display of the apparatus 600 may be used to initiate telepresence communications, may be used to request the diagnostic indication from the telehealth specialist, and may be used to receive the diagnostic indication from the telehealth specialist.

In some embodiments, the camera and/or the microphone may be disposed remotely from the computing device, either mounted within the medical transport or carried by one or more emergency medical personnel. The remotely disposed camera and/or microphone may allow for the camera and/or microphone to be placed in an optimal location to see or hear a patient in the medical transport or in location that the medical transport personnel prefers.

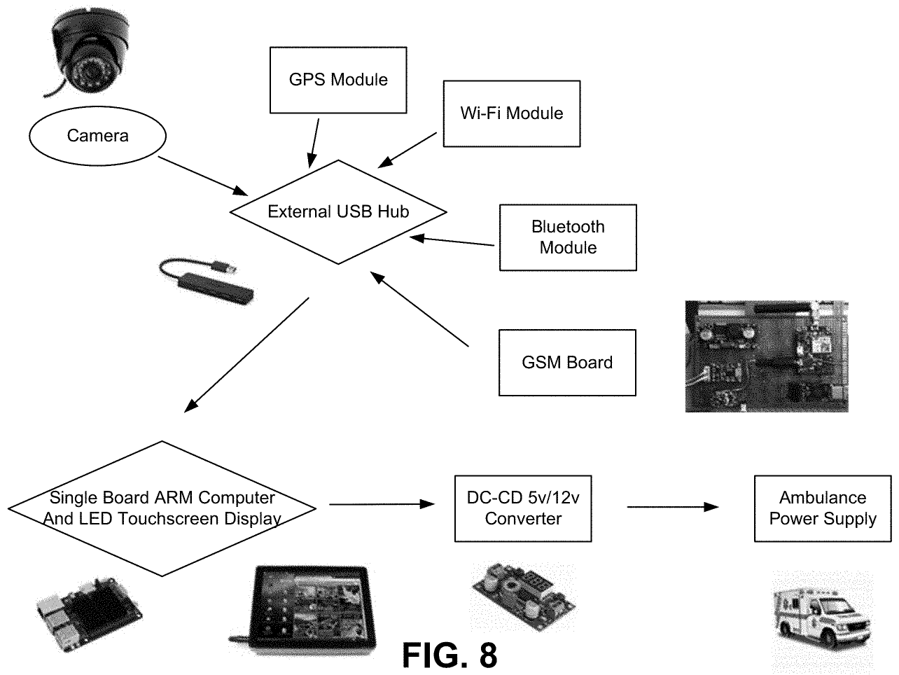

Referring now to FIG. 8, an example schematic diagram of various components in accordance with embodiments of the present invention is illustrated. Various components, including camera, GPS module, Wi-Fi module, Bluetooth module, and the GSM board, are in communication with the external USB hub.

The external USB hub in turn communicates with the single board ARM computer and LED touchscreen display. In some embodiments, the camera component is separated from the display and touchscreen component. In some embodiments, the LED touchscreen display is mounted closer to where EMTs are seated in the medical transport because they are not supposed to move from their seat once the vehicle is in motion. In some embodiments, the camera component is mounted at the back of the medical transport near the rear entry gate. This allows for fixed camera view towards the patient with minimal physical obstruction when entering/exiting the vehicle.

The single board ARM Computer and LED touchscreen display in turn communicates with a DC-CD 5 v/12 v converter, which is connected to the ambulance power supply.

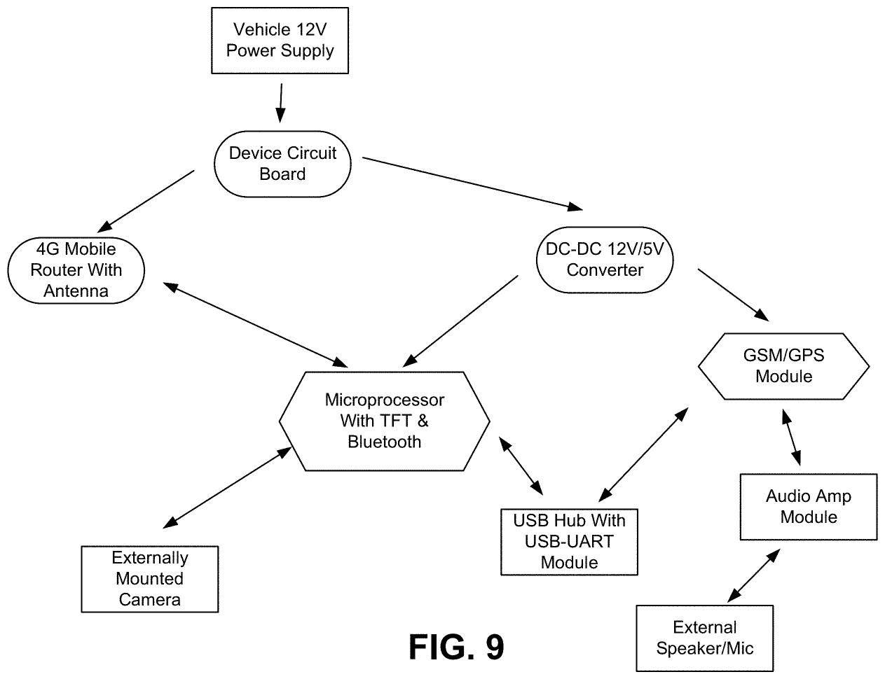

FIG. 9 illustrates an example schematic diagram of various components in accordance with embodiments of the present invention. As shown in FIG. 9, the Vehicle 12V Power Supply provides power to the Device Circuit Board. For example, Vehicle 12V Power Supply may be from the electrical system of a medical transport, and it may be used to provide power to the Device Circuit Board of an apparatus in accordance with various embodiments of the present invention.

The Device Circuit Board in turn transfers the power from the Vehicle 12V Power Supply to the DC-DC 12V/5V converter. The DC-DC 12V/5V converter may convert the 12V direct current to 5V direct current, which is used to power various components of the apparatus, such as the Microprocessor with Thin-Film-Transistor (TFT) & Bluetooth and the GSM/GPS Module.

The Microprocessor with TFT & Bluetooth provides processing functionalities, such as carrying out various calculations in methods described above in connection with FIGS. 3A-5. The Microprocessor with TFT & Bluetooth communicates with the 4G Mobile Router with Antenna, the Externally Mounted Camera, and the USB Hub with USB-UART Module. The 4G Mobile Router with Antenna is connected to the Device Circuit Board, and provides communications functions with a communications network, such as the communications network 107 described above with reference to FIG. 1. The Externally Mounted Camera provides imaging capturing functionalities, such as those described in connection with the one or more image capturing apparatus 115 of FIG. 1. The USB Hub with USB-UART Module provides expansion functions so that additional modules may be connected. For example, the GSM/GPS Module is connected to the USB Hub with USB-UART Module. The GSM/GPS Module provides navigation functions in addition to communications functions with a communications network (such as the communications network 107 described above with reference to FIG. 1).

An External Speaker/Mic is connected to the Audio AMP Module, which is in turn connected to the GSM/GPS Module. In various embodiments of the present invention, video audio signals (such as these described in connection with FIG. 5) may be captured by the External Speaker/Mic and transmitted via the GSM/GPS Module. The External Speaker/Mic may further output video audio signals received from the remote party.

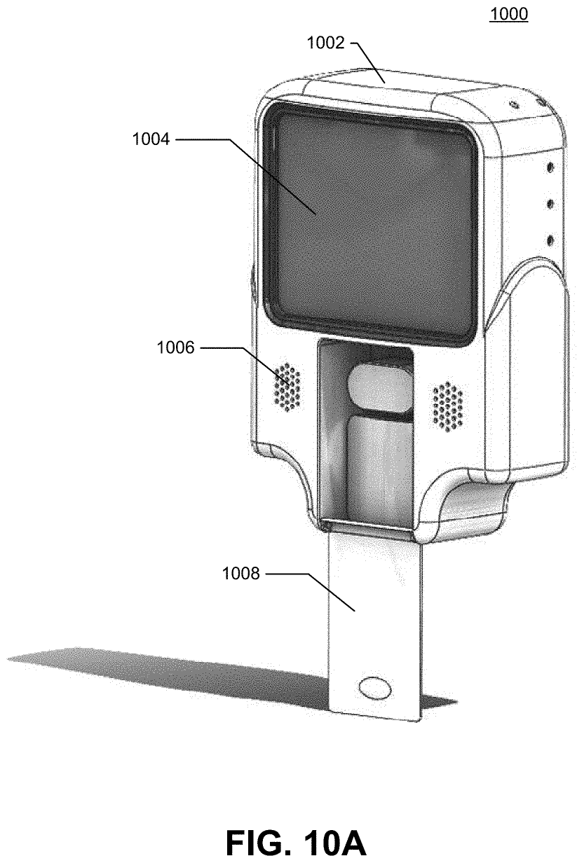

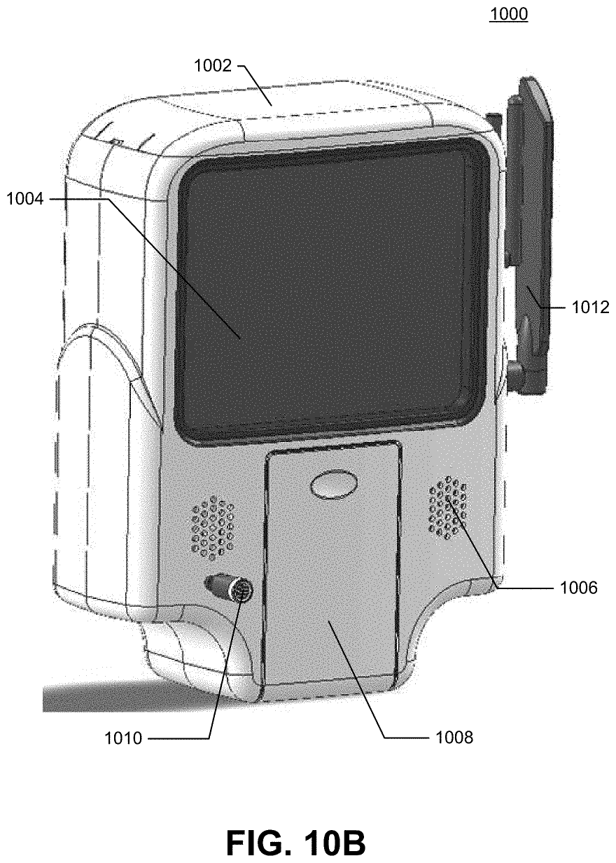

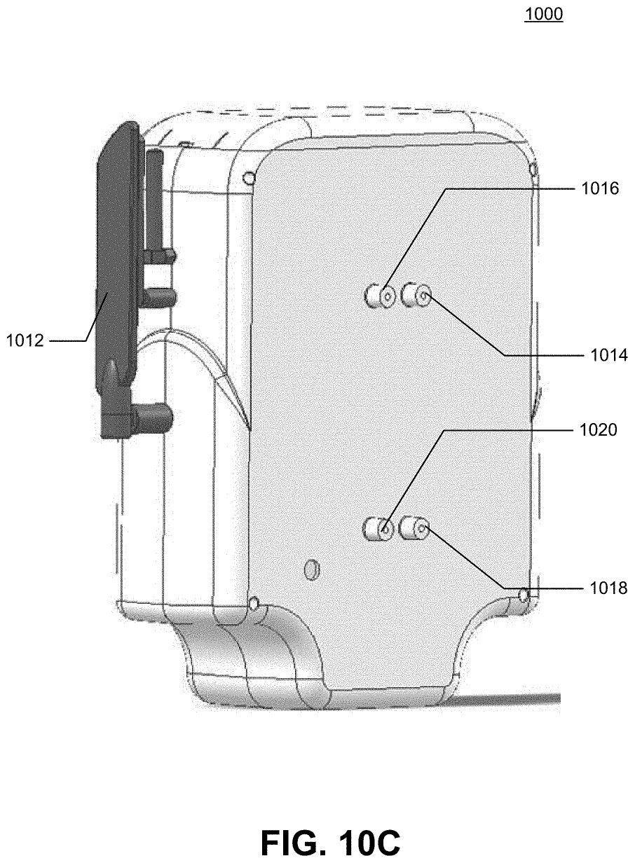

FIGS. 10A-10C show an apparatus 1000 that implements various embodiments of the present invention. The apparatus 1000 may be secured inside a medical transport, details of which are described hereinafter.

As shown in FIG. 10A, the apparatus 1000 may include a housing 1002, a display screen 1004, a mic/speaker unit 1006, and a flip cover 1008. The display screen 1004 is capable of displaying video images, such as video image signals from the remote party. The mic/speaker unit 1006 is capable of capturing the video audio signals inside the medical transport, and outputting received video audio signals from the remote party.

The housing 1002 provides space for a computer system (such as the computer system 109 described above with reference to FIGS. 1 and 2) so that various embodiments of the present invention may be carried out. For example, when an emergency medical personnel wants to start communicating with a remote party, the emergency medical personnel may initiate a communication through the apparatus 1000. As shown in FIG. 10B, the apparatus 1000 may further include an audio volume control knob 1010 so that the emergency medical personnel may control the output volume of the video audio. The apparatus 1000 may further include antenna 1012, which transmits and receives various signals (including the video audio signals and video image signals).

The apparatus 1000 may be mounted to an interior wall of a medical transport via the mounting points 1014, 1016, 1018, and 1020, as shown in FIG. 10C. In various embodiments of the present invention, fastener mechanisms are implemented via the mounting points 1014, 1016, 1018, and 1020 so that the apparatus 1000 can be securely attached to the medical transport.

### Additional Implementation Details

Although example processing systems have been described in the above mentioned figures, implementations of the subject matter and the functional operations described herein can be implemented in other types of digital electronic circuitry, or in computer software, firmware, or hardware, including the structures disclosed in this specification and their structural equivalents, or in combinations of one or more of them.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described herein can be implemented as one or more computer programs, i.e., one or more modules of computer program instructions, encoded on computer-readable storage medium for execution by, or to control the operation of, information/data processing apparatus. Alternatively, or in addition, the program instructions can be encoded on an artificially-generated propagated signal, e.g., a machine-generated electrical, optical, or electromagnetic signal, which is generated to encode information/data for transmission to suitable receiver apparatus for execution by an information/data processing apparatus. A computer-readable storage medium can be, or be included in, a computer-readable storage device, a computer-readable storage substrate, a random or serial access memory array or device, or a combination of one or more of them. Moreover, while a computer-readable storage medium is not a propagated signal, a computer-readable storage medium can be a source or destination of computer program instructions encoded in an artificially-generated propagated signal. The computer-readable storage medium can also be, or be included in, one or more separate physical components or media (e.g., multiple CDs, disks, or other storage devices).

The operations described herein can be implemented as operations performed by an information/data processing apparatus on information/data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (Application Specific Integrated Circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or information/data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described herein can be performed by one or more programmable processors executing one or more computer programs to perform actions by operating on input information/data and generating output. Processors suitable for the execution of a computer program include, by way of example, both general and special purpose microprocessors, and any one or more processors of any kind of digital computer. Generally, a processor will receive instructions and information/data from a read-only memory, a random access memory, or both. The essential elements of a computer are a processor for performing actions in accordance with instructions and one or more memory devices for storing instructions and data. Generally, a computer will also include, or be operatively coupled to receive information/data from or transfer information/data to, or both, one or more mass storage devices for storing data, e.g., magnetic, magneto-optical disks, or optical disks. However, a computer need not have such devices. Devices suitable for storing computer program instructions and information/data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described herein can be implemented on a computer having a display device, e.g., a CRT (cathode ray tube) or LCD (liquid crystal display) monitor, for displaying information/data to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described herein can be implemented in a computing system that includes a back-end component, e.g., as an information/data server, or that includes a middleware component, e.g., an application server, or that includes a front-end component, e.g., a client device having a graphical user interface or a web browser through which a user can interact with an implementation of the subject matter described herein, or any combination of one or more such back-end, middleware, or front-end components. The components of the system can be interconnected by any form or medium of digital information/data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits information/data (e.g., an HTML page) to a client device (e.g., for purposes of displaying information/data to and receiving user input from a user interacting with the client device). Information/data generated at the client device (e.g., a result of the user interaction) can be received from the client device at the server.

While this specification contains many specific implementation details, these should not be construed as limitations on the scope of any inventions or of what may be claimed, but rather as description of features specific to particular embodiments of particular inventions. Certain features that are described herein in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results, unless described otherwise. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results, unless described otherwise. In certain implementations, multitasking and parallel processing may be advantageous.

### Conclusion

Many modifications and other embodiments of the inventions set forth herein will come to mind to one skilled in the art to which these inventions pertain having the benefit of the teachings presented in the foregoing description and the associated drawings. Therefore, it is to be understood that the inventions are not to be limited to the specific embodiments disclosed and that modifications and other embodiments are intended to be included within the scope of the appended claims. For example, although several medical routing scenarios are described with corresponding apparatus, systems, and methods, the disclosure herein may also be applicable to other navigation, routing, and communication apparatus, systems, and methods. Although specific terms are employed herein, they are used in a generic and descriptive sense only and not for purposes of limitation, unless described otherwise.

That which is claimed:

1. A telepresence system for diagnosing and routing a patient comprising:
a medical transport at a first location;
a communications controller configured to connect the medical transport with a remote telehealth specialist;
an input device in the medical transport configured to receive at least one input, including a code identification, from medical transport personnel;
a microphone configured to generate a first audio signal and transmit the first audio signal to the communications controller;
a camera mounted in the medical transport, wherein the camera is configured to generate a first video signal and transmit the first video signal to the communications controller;
a navigation analyzer; and
at least one display disposed in the medical transport;
wherein the communications controller is configured to:
transmit the at least one input via at least one of a first wireless network and a second wireless network;
connect the medical transport to the remote telehealth specialist at a second location via each of the first wireless network and the second wireless network,
transmit the first video signal from the camera to the remote telehealth specialist via the first wireless network,
transmit the first audio signal from the microphone to the remote telehealth specialist via the second wireless network,
receive a second video signal from the remote telehealth specialist over the first wireless network;
transmit the second video signal to the at least one display in the medical transport;
receive a second audio signal from the remote telehealth specialist over the second wireless network;
transmit the second audio signal to at least one audio output device in the medical transport;
receive a diagnostic indication from the remote telehealth specialist via at least one of the first wireless network and the second wireless network; and
transmit the diagnostic indication to the navigation analyzer;
wherein the navigation analyzer is configured to:
receive at least one of the first location of the medical transport and a current location of the medical transport;
receive the diagnostic indication;
generate navigation data comprising navigation instructions from the at least one of the first location of the medical transport and the current location of the medical transport to an emergency department based on the diagnostic indication; and
transmit the navigation data to the at least one display in the medical transport.

2. The telepresence system of claim 1, wherein the code identification indicating the diagnosis of a patient is a code identification for a heart attack.

3. The telepresence system of claim 1, wherein the code identification indicating the diagnosis of a patient is a code identification for a cerebrovascular accident.

4. The telepresence system of claim 3, wherein the code identification for the cerebrovascular accident is related to a large vessel occlusion.

5. The telepresence system of claim 3, wherein the code identification for the cerebrovascular accident is not related to a large vessel occlusion.

6. The telepresence system of claim 1, wherein the navigation data to the emergency department is to an outpatient only facility.

7. The telepresence system of claim 1, wherein the navigation data to the emergency department is to an inpatient capable facility.

8. The telepresence system of claim 1, wherein the navigation data to the emergency department is to a non-invasive treatment facility.

9. The telepresence system of claim 1, wherein the navigation data to the emergency department is to an invasive treatment facility.

10. The telepresence system of claim 1, wherein the first wireless network and the second wireless network each comprises at least one of cellular signals, radio signals, or Wi-Fi signals.

11. The telepresence system of claim 1, wherein the emergency department comprises a home hospital network emergency department that is within the patient's home hospital network.

12. The telepresence system of claim 1, wherein the emergency department comprises a payor network emergency department that is within the patient's payor network.

13. A telepresence system for diagnosing and routing a patient comprising:
a medical transport at a first location;
a communications controller configured to connect the medical transport with a remote telehealth specialist;
an input device in the medical transport configured to receive at least one input, including a code identification, from medical transport personnel;
a microphone configured to generate a first audio signal and transmit the first audio signal to the communications controller;
a camera mounted in the medical transport, wherein the camera is configured to generate a first video signal and transmit the first video signal to the communications controller;
a navigation analyzer; and
at least one display disposed in the medical transport;
wherein the communications controller is configured to:
transmit the at least one input via at least one of a first wireless network and a second wireless network;
connect the medical transport to the remote telehealth specialist at a second location via each of the first wireless network and the second wireless network,
transmit the first video signal from the camera to the remote telehealth specialist via the first wireless network,
transmit the first audio signal from the microphone to the remote telehealth specialist via the second wireless network,
receive a second video signal from the remote telehealth specialist over the first wireless network;
transmit the second video signal to the at least one display in the medical transport;
receive a second audio signal from the remote telehealth specialist over the second wireless network;
transmit the second audio signal to at least one audio output device in the medical transport;
receive a diagnostic indication from the remote telehealth specialist via at least one of the first wireless network and the second wireless network; and
transmit the diagnostic indication to the navigation analyzer;
wherein the navigation analyzer is configured to:
receive at least one of the first location of the medical transport and a current location of the medical transport;
receive the code identification;
receive the navigation diagnostic indication;
generate navigation data comprising navigation instructions from the at least one of the first location of the medical transport and the current location of the medical transport to an emergency department based on the code identification and the diagnostic indication; and
transmit the navigation data to the at least one display in the medical transport.

14. The telepresence system of claim 13, wherein the code identification indicating the diagnosis of a patient is a code identification for a heart attack.

15. The telepresence system of claim 13, wherein the code identification indicating the diagnosis of a patient is a code identification for a cerebrovascular accident.

16. The telepresence system of claim 15, wherein the code identification for the cerebrovascular accident is related to a large vessel occlusion.

17. The telepresence system of claim 15, wherein the code identification for the cerebrovascular accident is not related to a large vessel occlusion.

18. The telepresence system of claim 13, wherein the emergency department comprises a home hospital network emergency department that is within the patient's home hospital network.

19. The telepresence system of claim 13, wherein the emergency department comprises a payor network emergency department that is within the patient's payor network.

20. A method for communicating between a telepresence system at a first location and a remote telehealth specialist at a second location, comprising:
generating a first video signal from a camera mounted in a medical transport at the first location;
generating a first audio signal from a microphone mounted in the medical transport;
receiving a code identification from an input device mounted in the medical transport;
transmitting the first video signal to the remote telehealth specialist at a separate remote location using a first wireless network;
transmitting the first audio signal to the remote telehealth specialist at a separate remote location using a second wireless network;
transmitting the code identification to the remote telehealth specialist via at least one of the first wireless network and the second wireless network;
receiving a second video signal over the first wireless network of the remote telehealth specialist from a camera at the remote location;
receiving a second audio signal over the second wireless network of from the remote telehealth specialist from a microphone at the remote location;
receiving a diagnostic indication from over either the first or second network from the remote telehealth specialist;
displaying the second video signal on a display in the medical transport;
playing the second audio signal over a speaker in the medical transport;
generating navigation data to an emergency department based on at least one of the code identification or the diagnostic indication; and
displaying the navigation data on the display in the medical transport.
